(19) United States
(12) Patent Application Publication
(10) Pub. No.: US 2020/0031735 A1
(43) Pub. Date: Jan. 30, 2020

(54) POLYFUNCTIONAL CATALYSTS

(71) Applicant: REGENTS OF THE UNIVERSITY OF MINNESOTA, Minneapolis, MI (US)

(72) Inventors: Anurag Kumar, Minneapolis, MN (US); Neil Razdan, Minneapolis, MN (US); Aditya Bhan, Minneapolis, MN (US)

(21) Appl. No.: 16/524,928

(22) Filed: Jul. 29, 2019

Related U.S. Application Data
(60) Provisional application No. 62/711,604, filed on Jul. 29, 2018.

(51) Int. Cl. C07C 2/78; B01J 29/48; B01J 35/02; B01J 37/08

(52) U.S. Cl. CPC C07C 2/78; B01J 29/48; B01J 35/023; B01J 2229/18; C07C 2529/48; C07C 2523/28; B01J 37/082

(57) ABSTRACT

Embodiments of the present disclosure describe catalysts, methods of preparing catalysts, methods of forming hydrocarbons using the catalysts, and the like.

### STATEMENT OF GOVERNMENT RIGHTS

This invention was made with government support under DE-SC0008418 awarded by the U.S. Department of Energy. The government has certain rights in the invention.

### BACKGROUND

Dehydroaromatization (DHA) provides an attractive thermochemical route for methane valorization in reference to indirect and oxidative routes that bring forth significant kinetic challenges in conferring high selectivity. The non-oxidative conversion of CH₄ by pyrolysis reactions to produce aromatics occurs with near equilibrium yield on carbidic forms of Mo and W and on metallic Re clusters encapsulated in zeolites at temperatures ˜950 K. The dehydroaromatization of methane to benzene (with ˜70% selectivity on a carbon basis) at these temperatures is restricted by equilibrium considerations (6CH₄↔C₆H₆+9H₂) to ˜10% single pass conversion with metal carbide clusters providing catalytic surfaces required for C—H bond activation and Brensted acid sites on the zeolite catalyzing carbon chain growth reactions. The net rate of C₂⁺ hydrocarbons formation decreases with decreasing space velocity and increasing conversion necessitated by the approach to equilibrium, however, forward rates of hydrocarbon and benzene synthesis as well as selectivity to benzene are noted to be invariant. The addition of hydrogen to influent mixtures of CH₄/Ar also results in lower net rates of hydrocarbon synthesis mandated by equilibrium, however, it has been shown that the forward rates of hydrocarbon and benzene synthesis are unperturbed by the addition of hydrogen demonstrating that hydrogen has no kinetic effect on the kinetically-relevant step of methane dehydroaromatization.

Oxidic precursors of Mo deposited either by aqueous phase impregnation methods or vapor phase solid state ion exchange result in formulations that exhibit similar characteristics for methane DHA. Spectroscopic features corresponding to bulk MoO₃ precursors are noted to disappear upon thermal treatment of MoO₃/HZSM-5 mixtures in air at 973 K and chemical titration and probe molecule spectroscopy studies including in-situ FT-IR spectroscopy, differential thermal analysis (DTA), NH₃-temperature-programmed-desorption (TPD), ²⁷Al MAS NMR, and H/D isotopic exchange demonstrate that some fraction of Brønsted acid sites in the zeolite are exchanged by MoOₓ species. This high temperature (˜973 K) surface migration of MoOₓ species is enabled by the lattice mobility within MoO₃ which is rendered feasible above its Tamman temperature (˜543 K). A 1:1 Mo/Al stoichiometry as probed via water desorption and D₂-OH exchange experiments is reported corresponding to exchange on Brensted acid sites. Iglesia and coworkers first reported that hexavalent Mo forms dimeric (Mo₂O₅)²⁺ species exchanged with two proximate Al centers by noting incomplete exchange of Mo and presence of residual protons in these samples, identifying Mo-speciation by X-ray absorption and UV-visible near infrared (UV-Vis) spectroscopy, and affirming stoichiometry of Mo₂O₅ moieties in chemical transient studies that noted the elution of ˜2.5 O:Mo upon reduction of the MoOₓ species.

These oxidic precursors are noted to disappear during an initial induction period that results primarily in the evolution of COₓ and H₂O without the concurrent formation of hydrocarbons. X-ray absorption, temperature-programmed-oxidation, isotopic exchange, and DME chemical titration studies show that MoCₓ clusters of 0.6-1 nm size are formed after carburization of (Mo₂O₅)²⁺ dimers in Mo/H-ZSM-5 catalyst and a fraction of the Brønsted acid sites, previously exchanged with MoOₓ dimers, are regenerated. The resulting MoCₓ clusters and their involvement in catalytic C—H bond activation during methane dehydroaromatization reactions at ˜973 K on Mo/H-ZSM-5 catalysts has been evidenced using X-ray photoelectron, ion sputtering, ⁹⁵Mo NMR, and infrared spectroscopy. Although the stoichiometry and coordination of MoCₓ clusters is still debated, the proficiency and reduced nature of active Mo-centers is no longer debated in the literature.

### SUMMARY

Polyfunctional catalysts, methods of non-oxidative methane dehydroaromatization using the polyfunctional catalysts, methods of preparing the polyfunctional catalysts, and the like are disclosed herein. The polyfunctional catalysts can comprise a pre-carburized catalyst and a hydrogen accepting component. The pre-carburized catalyst can be utilized to form, among other things, aromatics and hydrogen from methane. The hydrogen-accepting component can be utilized for in-situ hydrogen removal—forming, for example, metal hydrides—to overcome thermodynamic limitations thereby enhancing methane conversion and yield of aromatics. The polyfunctional catalysts can be provided in any of a variety of forms, such as staged and stratified catalyst beds and as interparticle mixtures, among other forms. Advantageously, the hydrogen-accepting component can easily be regenerated by thermal treatment under inert flow and reused in additional reaction cycles to enhance methane conversion and aromatic yields. These and other features of the invention are discussed in more detail herein.

In a first aspect, the present invention is directed to a method of producing aromatics, the method comprising contacting a methane-containing feed stream with a pre-carburized catalyst to form aromatics and hydrogen, wherein the pre-carburized catalyst is disposed within a reactor and comprises a carbidic form of an active metal supported on a proton form of a zeolite, wherein the reactor further comprises Zr particles that remove at least a portion of said hydrogen.

In a further aspect, the present invention is directed to a staged and stratified catalyst for non-oxidative methane dehydroaromatization to form aromatics and hydrogen from methane, the catalyst comprising alternating layers of a pre-carburized catalyst and a hydrogen-accepting component, wherein the pre-carburized catalyst comprises a carbidic form of an active metal supported on a proton form of a zeolite, wherein the hydrogen-accepting component absorbs at least a portion of said hydrogen.

In another aspect, the present invention is directed to an interparticle catalyst mixture for non-oxidative methane dehydroaromatization to form aromatics and hydrogen from methane, the catalyst comprising: a mixture comprising particles of a pre-carburized catalyst and particles of a hydrogen-accepting component, wherein the pre-carburized catalyst comprises a carbidic form of an active metal supported on a proton form of a zeolite, wherein the hydrogen-accepting component absorbs at least a portion of said hydrogen.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIGS. 1A-1D are schematic diagrams of polyfunctional catalysts, according to one or more embodiments of the present disclosure.

FIG. 2 is a flowchart of a method of preparing a catalyst, according to one or more embodiments of the present disclosure.

FIG. 3 is a flowchart of a method of forming hydrocarbons, according to one or more embodiments of the present disclosure.

FIGS. 4A-4B show transient product formation rates and methane conversion as a function of time-on-stream for Mo/H-ZSM-5 at 973 K, total flow rate ˜0.21 cm³ s⁻¹ (90 vol % CH₄/balance Ar), and catalyst loading ˜1.2 g with Mo/Al_{f}˜0.25: (a) Symbols are GC data and lines are mass spectrometer (MS) transient data; (b) Symbols are GC data and lines are included as a guide to the eye, according to one or more embodiments of the present disclosure.

FIGS. 5A-5C show Raman spectra of (a) physical mixture of MoO₃ and H-ZSM-5 with Mo/Al_{f}˜0.25, (b) Mo/H-ZSM-5 with Mo/Al_{f}˜0.25, and (c) H-ZSM-5 treated in flowing air at 973 K for 5 h, where the lines shown are a guide to the eye for bands at 376 cm⁻¹, 820 cm⁻¹, and 970 cm⁻¹, according to one or more embodiments of the present disclosure.

FIG. 6 shows product formation rates and methane conversion as a function of time-on-stream for Mo/H-ZSM-5 at 973 K, total flow rate ˜0.21 cm³ s⁻¹ (90 vol % CH₄/balance Ar), and catalyst loading ˜1.2 g with Mo/Al_{f}˜0.25, where the dotted red line indicates the equilibrium conversion ˜10% for 6CH₄+↔C₆H₆+9H₂ at 973 K, according to one or more embodiments of the present disclosure.

FIG. 7 shows transient product formation rates and methane conversion as a function of time-on-stream for Mo/H-ZSM-5 and MoCₓ/H-ZSM-5 at 973 K, total flow rate ˜0.21 cm³ s⁻¹ (90 vol % CH₄/balance Ar), and catalyst loading ˜1.2 g with Mo/Al_{f}˜0.25. CH₄ reaction on Mo/H-ZSM-5 was stopped after 15.5 ks and the feed was switched to helium (˜0.83 cm³ s⁻¹) (The catalyst was cooled to room temperature, then heated to ˜973 K in the same helium flow, and finally CH₄ reaction was started on the pre-carburized catalyst designated as MoCₓ/H-ZSM-5. The dotted red line indicates the equilibrium conversion ˜10% for 6CH₄↔C₆H₆+9H₂ at 973 K. Symbols are GC data and lines are included as a guide to the eye), according to one or more embodiments of the present disclosure.

FIG. 8 shows methane effluent flow rate as a function of time-on-stream through bypass and Zr particles to test activity of zirconium metal for methane conversion (reaction at 973 K, total flow rate ˜0.21 cm³ s⁻¹ (90 vol % CH₄/balance Ar), and catalyst loading ˜2.4 g Zr metal; the line indicates the switch of flow from bypass to Zr particles (at 973 K)), according to one or more embodiments of the present disclosure.

FIGS. 9A-9B show transient (a) product formation rates and methane conversion, (b) effluent hydrogen rate and methane conversion, as a function of time-on-stream for an interpellet physical mixture of MoCₓ/H-ZSM-5 and Zr metal (Reaction conditions: temperature ˜973 K, total flow rate ˜0.21 cm³ s⁻¹ (90 vol % CH₄/balance Ar), and catalyst loading ˜1.2 g MoCₓ/H-ZSM-5 with Mo/Al_{f}˜0.25 and ˜2.4 g Zr metal. Symbols are GC data and lines are mass spectrometer (MS) transient data. The dotted red line indicates the equilibrium conversion ˜10% for 6CH₄↔C₆H₆+9H₂ at 973 K), according to one or more embodiments of the present disclosure.

FIGS. 10A-10F show transient (a) methane conversion, (b) C₂Hₓ, (c) benzene, (d) naphthalene, (e) toluene, and (f) hydrogen formation rates as a function of time-on-stream for MoCₓ/H-ZSM-5 and an interpellet physical mixture of MoCₓ/H-ZSM-5 and Zr metal (MoCₓ/H-ZSM-5 loading ˜1.2 g with Mo/Al_{f}˜0.25, Zr metal loading ˜2.4 g, total flow rate ˜0.21 cm³ s⁻¹ (90 vol % CH₄/balance Ar), reaction at ˜973 K. The dotted red line in (a) indicates the equilibrium conversion ˜10% for 6CH₄↔C₆H₆+9H₂ at 973 K), according to one or more embodiments of the present disclosure.

FIGS. 11A-11G show cumulative (a) methane converted, (b) benzene, (c) naphthalene, (d) toluene, (e) xylenes, (f) C₁₀⁺, and (g) C₂Hₓ yield as a function of time-on-stream for MoCₓ/H-ZSM-5 and an interpellet physical mixture of MoCₓ/H-ZSM-5 and Zr metal (MoCₓ/H-ZSM-5 loading ˜1.2 g with Mo/Al_{f}˜0.25, Zr metal loading ˜2.4 g, total flow rate ˜0.21 cm³ s⁻¹ (90 vol % CH₄/balance Ar), reaction at ˜973 K, according to one or more embodiments of the present disclosure.

FIGS. 12A-12C show (a) Normalized Ar (m/z=40) and H₂ (m/z=2) effluent flow rates obtained using a mass spectrometer at different hydrogen feed pressures (3.28-95.13 kPa) during hydrogen uptake experiments for Zr metal; (b) Normalized H₂ effluent flow rate and reactor temperature during temperature-programmed-desorption (TPD) in helium flow (˜0.83 cm³ s⁻¹) at ˜1193 K, following the hydrogen uptake shown in FIG. 12A; (c) Hydrogen to zirconium molar ratio as a function of hydrogen uptake pressure calculated from hydrogen uptake (FIG. 12A) (H_{absorbed}:Zr) and helium TPD (FIG. 12B) (H_{desorbed}:Zr). Zr metal loading ˜2.4 g, Zr particle diameter ˜3×10⁻⁴ m, total feed flow rate ˜1.7 cm³ s⁻¹, H₂/Ar˜(3.28-95.13) kPa/balance, temperature ˜973 K, according to one or more embodiments of the present disclosure.

FIG. 13 show normalized Ar (m/z=40) and H₂ (m/z=2) effluent flow rates obtained using a mass spectrometer through a blank reactor (Total feed flow rate ˜1.7 cm³ s⁻¹, H₂/Ar˜95.13 kPa/balance, temperature ˜973 K), according to one or more embodiments of the present disclosure.

FIGS. 14A-14B show X-ray diffraction patterns of (a) Zr metal, and (b) Zr hydride formed by hydrogen uptake of Zr metal as shown in FIG. 9A (The reference diffraction patterns are also included for Zr metal (JCPDS PDF #03-065-3366) in (a) and ZrH_{1.66} (JCPDS PDF #00-034-0649) and ZrH (JCPDS PDF #00-034-0690) in (b)), according to one or more embodiments of the present disclosure.

FIG. 15 show mass spectrometer transient of H₂ (m/z=2) effluent flow rate during helium flush and temperature-programmed-desorption (TPD) in helium flow (˜0.83 cm³ s⁻¹) at ˜1193 K, following methane reaction on interpellet physical mixture of MoCₓ/H-ZSM-5 and Zr metal, shown in FIG. 16 (Regenerations 1, 2, and 3 of Zr+MoCₓ/H-ZSM-5 were performed by flushing the catalyst in helium flow (˜0.83 cm³ s⁻¹) at 973 K for 61.2 ks, 84.6 ks, and 34.2 ks respectively. MoCₓ/H-ZSM-5 loading ˜1.2 g with Mo/Al_{f}˜0.25, Zr metal loading ˜2.4 g, total flow rate ˜0.21 cm³ s⁻¹ (90 vol % CH₄/balance Ar), reaction at ˜973 K), according to one or more embodiments of the present disclosure.

FIG. 16 show transient methane conversion as a function of time-on-stream for MoCₓ/H-ZSM-5 and for an interpellet physical mixture of MoCₓ/H-ZSM-5 and Zr metal before (fresh) and after regeneration in helium flow (Regenerations 1, 2, and 3 of Zr+MoCₓ/H-ZSM-5 were performed by flushing the catalyst in helium flow (˜0.83 cm³ s⁻¹) at 973 K for 61.2 ks, 84.6 ks, and 34.2 ks respectively. MoCₓ/H-ZSM-5 loading ˜1.2 g with Mo/Al_{f}˜0.25, Zr metal loading ˜2.4 g, total flow rate ˜0.21 cm³ s⁻¹ (90 vol % CH₄/balance Ar), reaction at ˜973 K. The dotted red line indicates the equilibrium conversion ˜10% for 6CH₄+↔C₆H₆+9H₂ at 973 K), according to one or more embodiments of the present disclosure.

FIG. 17 show mass spectrometer transient of H₂ (m/z=2) and methane (m/z=16) effluent flow rate and reactor temperature during temperature-programmed-desorption (TPD) in helium flow (˜0.83 cm³ s⁻¹) at ˜1193 K, following methane reaction on interpellet physical mixture of MoCₓ/H-ZSM-5 and Zr metal, shown in FIGS. 9A-9B (Reaction conditions: temperature ˜973 K, total flow rate ˜0.21 cm³ s⁻¹ (90 vol % CH₄/balance Ar), and catalyst loading ˜1.2 g MoCₓ/H-ZSM-5 with Mo/Al_{f}˜0.25 and ˜2.4 g Zr metal), according to one or more embodiments of the present disclosure.

FIGS. 18A-18D are graphical views showing transient (a) methane conversion, (b) benzene, (c) naphthalene formation rates as a function of time-on-stream for all five studied configurations, where the dashed line in (a) indicates the equilibrium conversion ˜10% for 6CH₄↔C₆H₆+9H₂ at 973 K and 1 atmosphere total pressure, and (d) maximum single-pass methane conversion (right axis) and cumulative product yield at 10.2 ks time-on-stream (left axis) on MoCₓ/H-ZSM-5 and Zr catalyst beds configured with (i) MoCₓ/H-ZSM-5 only, (ii) Zr packed upstream of MoCₓ/H-ZSM-5, (iii) Zr packed downstream of MoCₓ/H-ZSM-5, (iv) Zr packed both upstream and downstream of MoCₓ/H-ZSM-5, and (v) an interpellet physical mixture of Zr and MoCₓ/H-ZSM-5 (lines are to guide the eye) (Reaction conditions: 973 K, MoCₓ/H-ZSM-5≈1.2 g, Zr≈2.4 g, feed flow rate≈0.21 cm³ s⁻¹ (90 vol % CH₄), according to one or more embodiments of the present disclosure.

FIG. 19 is a graphical view showing effluent flow rates during TPD in ˜0.83 cm³ s⁻¹ He flow at ˜1193 K post-CH₄ reaction on an interpellet Zr+MoCₓ/H-ZSM-5 mixture for ˜9 ks, according to one or more embodiments of the present disclosure.

FIG. 20 is a graphical view showing hydrogen missing during methane DHA reaction (black data set) and hydrogen desorbed during post-reaction helium TPD at 1193 K (red data set) for MoCₓ/H-ZSM-5 and Zr catalyst beds configured with Zr packed downstream of MoCₓ/H-ZSM-5 (Zr after), Zr packed upstream of MoCₓ/H-ZSM-5 (Zr before), Zr packed both upstream and downstream of MoCₓ/H-ZSM-5 (sandwich), and an interpellet physical mixture of Zr and MoCₓ/H-ZSM-5. MoCₓ/H-ZSM-5 loading ˜1.2 g with Mo/Al_{f}˜0.25, Zr metal loading ˜2.4 g, total flow rate ˜0.21 cm³ s⁻¹ (90 vol % CH₄/balance Ar), reaction at ˜973 K, according to one or more embodiments of the present disclosure.

FIGS. 21A-21C are graphical views showing cumulative (a) methane converted, (b) benzene and (c) naphthalene yield as a function of time-on-stream on MoCₓ/H-ZSM-5 and Zr catalyst beds configured with MoCₓ/H-ZSM-5 only, Zr packed downstream of MoCₓ/H-ZSM-5, Zr packed upstream of MoCₓ/H-ZSM-5, Zr packed both upstream and downstream of MoCₓ/H-ZSM-5 (sandwich), and an interpellet physical mixture of Zr and MoCₓ/H-ZSM-5. MoCₓ/H-ZSM-5 loading ˜1.2 g with Mo/Al_{f}˜0.25, Zr metal loading ˜2.4 g, total flow rate ˜0.21 cm³ s⁻¹ (90 vol % CH₄/balance Ar), reaction at ˜973 K, according to one or more embodiments of the present disclosure.

FIG. 22 is a graphical view showing cumulative product selectivity (carbon basis) calculated at 10.2 ks time-on-stream on MoCₓ/H-ZSM-5 and Zr catalyst beds configured with MoCₓ/H-ZSM-5 only (No Zr), Zr packed downstream of MoCₓ/H-ZSM-5, Zr packed upstream of MoCₓ/H-ZSM-5, Zr packed both upstream and downstream of MoCₓ/H-ZSM-5 (sandwich), and an interpellet physical mixture of Zr and MoCₓ/H-ZSM-5. MoCₓ/H-ZSM-5 loading ˜1.2 g with Mo/Al_{f}˜0.25, Zr metal loading ˜2.4 g, total flow rate ˜0.21 cm³ s⁻¹ (90 vol % CH₄/balance Ar), reaction at ˜973 K, according to one or more embodiments of the present disclosure.

FIGS. 23A-23B are graphical views showing the effect of H₂ partial pressure (solid black diamonds, varied from 0 to 23 kPa at 97.6 kPa CH₄ and balance argon, total flow rate ca. 0.27 cm³ s⁻¹, temperature=973 K and catalyst loading=1.2 g) and methane partial pressure (solid black circles, varied from 2.7 to 97.6 kPa, total flow rate ca. 0.27 cm³ s⁻¹, temperature=973 K and catalyst loading=1.2 g) on (a) forward benzene synthesis rate and (b) forward naphthalene synthesis rate, where methane conversion was in the range 1.2-8.1% (dashed lines are included as a guide to the eye), according to one or more embodiments of the present disclosure.

FIGS. 24A-24D are graphical views showing non-dimensional tracer response curves (• or —) per effluent H₂ content monitored by online mass spectrometer and ideal response fit (—) upon introduction of H₂ by (a) impulse at 973 K with 0.33 cm³ s⁻¹ total flow and 0.68 cm reactor diameter, (b) impulse at 973 K with 0.66 cm³ s⁻¹ total flow and 0.68 cm reactor diameter, (c) step-change at 303 K with 0.29 cm³ s⁻¹ total flow and 1.05 cm reactor diameter, and (d) step-change at 973 K with 0.29 cm³ s⁻¹ total flow and 1.05 cm reactor diameter, where solid black lines in 3(a) and 3(b) are to guide the eye, according to one or more embodiments of the present disclosure.

FIG. 25 is a graphical view showing Cₚᵤₗₛₑ response curve from impulse input of H₂ at 973 K with 0.33 cm³ s⁻¹ total flow and a 0.68 cm diameter reactor packed with sufficient quartz sand (mesh 40-80) to result in 2.7 cm of bed, according to one or more embodiments of the present disclosure.

FIGS. 26A-26B are graphical views showing (a) Hydrogen partial pressure and (b) maximum single-pass (steady-state) methane conversion along non-dimensional axial coordinate of catalyst bed, x, for an ideal plug flow reactor with Pe=100 as predicted by simulation of the kinetic-transport model for MoCx/H-ZSM-5 only (No Zr) and Zr packed downstream of MoCₓ/H-ZSM-5 (Reaction conditions: 973 K, MoCₓ/H-ZSM-5≈1.2 g, Zr≈2.4 g, feed flow rate≈0.21 cm³ s⁻¹ (90 vol % CH₄)), according to one or more embodiments of the present disclosure.

FIGS. 27A-27B are graphical views showing (a) maximum single-pass (steady-state) (benzene+naphthalene) yield and (b) P_{H2} along non-dimensional axial coordinate of catalyst bed, x, for all studied reaction configurations as predicted by simulation of the kinetic-transport model (lines) and quantified using the reactor effluent during methane DHA reactions (symbols) (reaction conditions: 973 K, MoCₓ/H-ZSM-5≈1.2 g, Zr≈2.4 g, feed flow rate≈0.21 cm³ s⁻¹ (90 vol % CH₄), according to one or more embodiments of the present disclosure.

FIG. 28 is a graphical view showing a surface plot of maximum single-pass B+N yield as a function of Da_{B} and Pe with downstream Zr bed per simulation of the reaction-transport model (The red curve is the path parameterized by change of linear velocity, u. The black line is the path parameterized by change of bed length, L. The projection of the surface plot onto the red curve is shown as a dashed line in FIG. 30A of the main text. The projection of the surface plot onto the black line is shown as a dashed line in FIG. 30B of the main text), according to one or more embodiments of the present disclosure.

FIG. 29 is a graphical view showing a surface plot of maximum single-pass B+N yield as a function of Da_{B} and Pe without Zr per simulation of the reaction-transport model (The red curve is the path parameterized by change of linear velocity, u. The black line is the path parameterized by change of bed length, L. The projection of the surface plot onto the red curve is shown as a solid line in FIG. 30A of the main text. The projection of the surface plot onto the black line is shown as a solid line in FIG. 30B of the main text.), according to one or more embodiments of the present disclosure.

FIGS. 30A-30B are graphical views showing maximum single-pass (benzene+naphthalene) yield with and without Zr packed downstream of MoCₓ/H-ZSM-5 as measured (∘, •) and predicted by simulation of the reaction-transport model (—, —), as a function of catalyst bed length, L, and linear feed velocity, u; data labels I, II, and III refer to pairs of data at identical u/u₀ or L/L₀ (reaction conditions: 973 K, MoCₓ/H-ZSM-5≈(0.15-1.9) g, Zr≈2.4 g, feed flow rate≈(0.21-2.00) cm³ s⁻¹, L₀≈2.7 cm, u₀≈0.71 cm s⁻¹, according to one or more embodiments of the present disclosure.

FIGS. 31A-31D are graphical views showing instantaneous net benzene synthesis rate (left y-axes) and hydrogen effluent rate (right y-axes) as a function of time-on-stream during methane DHA reaction for (a) an interpellet physical mixture of Zr and MoCₓ/H-ZSM-5, (b) Zr packed downstream of MoCₓ/H-ZSM-5, (c) Zr packed upstream of MoCₓ/H-ZSM-5, and (d) sandwich configuration, compared to MoCₓ/H-ZSM-5 only in each case. Reaction conditions: 973 K, MoCₓ/H-ZSM-5≈1.2 g, Zr≈2.4 g, feed flow rate≈0.21 cm³ s⁻¹ (90 vol % CH₄), where dashed lines are linear fits for the curves as discussed in text and corresponding data shown in Table 2, according to one or more embodiments of the present disclosure.

### DETAILED DESCRIPTION

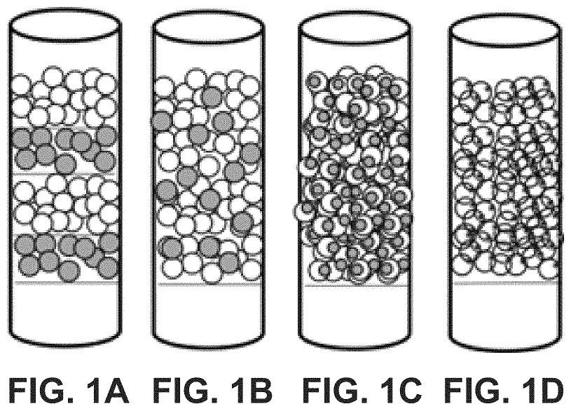

The invention of the present disclosure relates to catalysts and/or pre-catalysts for, among other things, methane dehydroaromatization with transient absorptive removal of hydrogen. In particular, the invention of the present disclosure relates to catalysts and/or pre-catalysts that increase yields of desired products in non-oxidative methane dehydrogenation on polyfunctional formulations (e.g., catalysts and/or pre-catalysts) by introducing an additional function that scavenges byproduct hydrogen thereby lifting thermodynamic constraints on attainable yields and increasing rates of products formation. The use of polyfunctional formulations (e.g., catalysts and/or pre-catalysts) enhance yield of aromatic products in methane conversion over, for example, carbidic forms of metal (e.g., Mo and W, among others) catalysts supported on porous supports (e.g., such as proton-form zeolites). Staged and stratified catalyst beds, and interparticle mixtures, among others, may be used. See, for example, FIGS. 1A-1D for schematic representations of each. Treatment (e.g., thermal treatment) in inert flows may be used to regenerate the absorbent function of the catalysts and/or pre-catalysts, among other things. Among other benefits of the present invention, this is the first disclosure of industrial processes that convert methane and/or natural gas feedstock to aromatics in a single-stage catalytic process.

For example, non-oxidative conversion of methane by pyrolysis reactions to produce aromatics may occur with near equilibrium yield on carbidic forms of molybdenum encapsulated in proton form zeolites at temperatures ˜700° C. The dehydroaromatization of methane to benzene (with ˜70% selectivity on a carbon basis) at these temperatures may be restricted by equilibrium considerations (6CH₄↔C₆H₆+9H₂) to ˜10% single pass conversion due to abundant hydrogen formed in the catalyst bed. In-situ removal of hydrogen by addition of zirconium reported herein may be used as an effective strategy to overcome these thermodynamic restrictions on single pass conversion. The improvement in single pass conversion of methane and yield of aromatics may be attained without any deleterious effects on selectivity to desirable products. The absorbent function can be regenerated by thermal treatment in inert flow and the regenerated absorbent can be used to attain higher than 10% conversion in successive reaction-regeneration cycles.

Embodiments of the present disclosure describe a catalyst comprising a first component and a second component. The first component may include a hydrogen-accepting component. The second component may include a porous support and an active metal on a surface and/or pores of the porous support. In other embodiments, the first component may include a porous support and an active metal on a surface and/or pores of the porous support; and the second component may include a hydrogen-accepting component.

The first component and the second component may be arranged in any of a variety of configurations (e.g., staged and/or stratified catalyst beds, interparticle mixtures, intraparticle mixtures, etc.). In many embodiments, the first component and the second component are arranged such that they are in physical contact, or immediate or close proximity. For example, in an embodiment, the polyfunctional catalyst may be provided as alternating stratified layers of a first component and a second component. In an embodiment, the polyfunctional catalyst may be provided as interparticle physical mixtures of a first component and a second component. In an embodiment, the polyfunctional catalyst may be provided as intraparticle physical mixtures of a first component and a second component. In an embodiment, the polyfunctional catalyst may be provided in which the first component or second component encapsulates the other component (e.g., the second component or first component, respectively).

The hydrogen-accepting component may include any element, compound, and/or complex capable of accepting a hydrogen. For example, the hydrogen-accepting component may include any element, compound, and/or complex capable of accepting and/or bonding to a hydrogen to form a hydride. The hydrogen-accepting component may include at least one or more of alkali metals, alkaline earth metals, transition metals, and metalloids. In many embodiments, the hydrogen-accepting component includes at least transition metals. The transition metals may include, but are not limited to, one or more of zirconium, titanium, niobium, tantalum, hafnium, vanadium, and zinc. In preferred embodiments, the hydrogen-accepting component includes zirconium. The hydrogen-accepting component may be provided in any form, such as particles and/or powders. For example, a particle size of the hydrogen-accepting component may range from about 180 μm to about 425 μm. In other embodiments, a particle size of the hydrogen-accepting component may be less than about 180 μm and/or greater than about 425 μm.

The active metal may include any metal that exhibits catalytic activity upon being contacting with a fluid composition containing methane (e.g., under methane dehydroaromatization conditions, non-oxidative conversion of methane, etc.). For example, the active metal may include one or more of molybdenum, vanadium, chromium manganese, zinc, iron, cobalt, nickel, copper, gallium, germanium, niobium, molybdenum, ruthenium, rhodium, silver, tantalum, tungsten, rhenium, platinum, and lead. In many embodiments, the active metal includes a carbidic form of the active metal. For example, in a preferred embodiment, the active metal includes a carbidic form of molybdenum. In another preferred embodiment, the active metal includes a carbidic form of tungsten. In an embodiment, the pre-carburized catalyst comprises an active metal of the formula MCₓ, where M is the active metal, C is carbon, and x is at least 0.01.

The porous support may include any porous material. In many embodiments, the porous support includes an inorganic oxide support. For example, the porous support may include one or more of zeolites, non-zeolitic molecular sieves, silica, alumina, zirconia, titania, yttria, ceria, and rare earth metal oxides. In many embodiments, the porous support includes zeolites, preferably a proton form of zeolites. The zeolites may include one or more of ZSM-5, ZSM-8, ZSM-11, ZSM-12, ZSM-22, and ZSM-35. In a preferred embodiment, the porous support includes ZSM-5. In a more preferred embodiment, the porous support includes a proton form of ZSM-5.

In an embodiment, the polyfunctional catalyst comprises a first component and a second component, wherein the first component includes a zirconium particle and the second component includes a proton form of a zeolite and a carbidic form of Mo or W on a surface and/or pores of the proton form of the zeolite.

In an embodiment, the polyfunctional catalyst is provided as a staged and stratified catalyst for non-oxidative methane dehydroaromatization to form aromatics and hydrogen from methane. For example, in certain embodiments, the staged and stratified catalyst comprises alternating layers of a pre-carburized catalyst and a hydrogen-accepting component, wherein the pre-carburized catalyst comprises a carbidic form of an active metal supported on a proton form of a zeolite, wherein the hydrogen-accepting component absorbs at least a portion of said hydrogen.

In an embodiment, the hydrogen-accepting component is positioned upstream from the pre-carburized catalyst. In an embodiment, the hydrogen-accepting component is positioned downstream from the pre-carburized catalyst. In an embodiment, the hydrogen-accepting component is positioned both downstream and upstream from the pre-carburized catalyst.

In an embodiment, a polyfunctional catalyst is provided in a continuous flow configuration. For example, in certain embodiments, the polyfunctional catalyst comprises alternating layers of a pre-carburized catalyst and a hydrogen-accepting component, wherein the hydrogen-accepting component continuously flows (e.g., as a moving bed of particles) to a regeneration unit, wherein accepted or absorbed hydrogen is removed or released from the hydrogen-accepting component and the hydrogen-accepting component is regenerated to full hydrogen-storage capacity. The hydrogen-accepting component can be thus be regenerated and reused in one or more reaction cycles.

In an embodiment, a polyfunctional catalyst is provided in a fluidized bed reactor, wherein the discrepancy in density of the two components is leveraged to separate and regenerate each component cyclically. For example, in certain embodiments, the pre-carburized catalyst and hydrogen-accepting component can be separated based on differences or discrepancies in density.

In an embodiment, the polyfunctional catalyst is provided as an interparticle mixture for non-oxidative methane dehydroaromatization to form aromatics and hydrogen from methane. For example, in certain embodiments, the interparticle mixture or interparticle catalyst mixture comprises a mixture comprising particles of a pre-carburized catalyst and particles of a hydrogen-accepting component, wherein the pre-carburized catalyst comprises a carbidic form of an active metal supported on a proton form of a zeolite, wherein the hydrogen-accepting component absorbs at least a portion of said hydrogen.

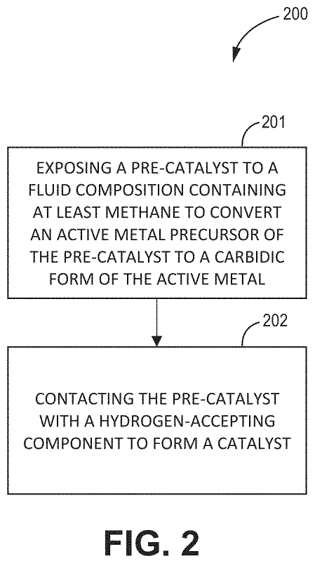

FIG. 2 is a flowchart of a method of preparing a catalyst, according to one or more embodiments of the present disclosure. As shown in FIG. 2, the method may comprise one or more of the following steps: exposing 201 a pre-catalyst to a fluid composition containing at least methane to convert an active metal precursor of the pre-catalyst to a carbidic or carburized form of the active metal; and contacting 202 the pre-catalyst with a hydrogen-accepting component to form a catalyst.

The step 201 includes exposing a pre-catalyst to a fluid composition containing at least methane to convert an active metal precursor of the pre-catalyst to a carbidic form of the active metal. The exposing may include bringing the pre-catalyst and the fluid composition containing at least methane into physical contact, or immediate or close proximity. The exposing may proceed under heating and/or thermal treatment. For example, in an embodiment, the exposing may proceed at a temperature greater than about 600 K. In many embodiments, the exposing may proceed at a temperature greater than about 900 K. In preferred embodiments, the exposing may proceed at about 973 K.

The pre-catalyst may include any of the porous supports, active metals, and/or precursors thereof described herein. For example, the pre-catalyst may include an active metal precursor on a surface of the pre-catalyst and/or in pores of the pre-catalyst. The active metal precursor may include any active metal compound. In an embodiment, the active metal precursor may include metal oxides, such as MoO₃, and/or dimers, such as (Mo₂O₅)⁺² on a surface and/or pores of the pre-catalyst. Upon the exposing of the pre-catalyst to the fluid composition containing at least methane, the active metal precursor may be carburized to form a carbidic form of the active metal. For example, in embodiments in which the active metal precursor includes one or more of MoO₃ and (Mo₂O₅)⁺² on a surface and/or pores of the pre-catalyst, a carbidic form of the active metal may be characterized by the chemical formula MoCₓ. The carbidic form of the active metal may be catalytically active. While the discussion above relates to Mo, any of the active metals of the present disclosure may be used herein, such as W, among others.

The step 202 includes contacting the pre-catalyst with a hydrogen-accepting component to form a catalyst. The pre-catalyst and hydrogen-accepting component may be contacted in a manner sufficient to form a catalyst in any of a variety of configurations (e.g., staged and/or stratified catalyst beds, interparticle mixtures, intraparticle mixtures, etc.). For example, the contacting may include bringing the pre-catalyst and the hydrogen-accepting component into physical contact, or immediate or close proximity. In an embodiment, the contacting may include mixing to form one or more of interpellet physical mixtures, interparticle physical mixtures, and intraparticle physical mixtures of the pre-catalyst and hydrogen-accepting component. In an embodiment, the contacting may include arranging the pre-catalyst and hydrogen-accepting component to provide a catalyst with alternating stratified layers of the pre-catalyst and hydrogen-accepting component. In an embodiment, the contacting may include encapsulating the pre-catalyst with the hydrogen-accepting component and/or encapsulating the hydrogen-accepting component with the pre-catalyst. These shall not be limiting as the contacting may proceed to form catalysts in other configurations.

The pre-catalyst may include any of the pre-catalysts of the present disclosure. For example, in many embodiments, the pre-catalyst may include a carbidic form of the active metal on a surface and/or pores of a porous support. For example, the pre-catalyst may include MoCₓ on a surface and pores of a porous support, such as H-ZSM-5, where x is at least 0.01. In other embodiments, the pre-catalyst may include an active metal precursor on a surface of the pre-catalyst and/or in pores of the pre-catalyst. The pre-catalyst may be characterized by a particle size ranging from about 180 μm to about 425 μm. In certain embodiments, the pre-carburized catalyst has an average particle size in the range of about 180 μm to about 425 μm. The hydrogen-accepting component may include any of the hydrogen-accepting components described herein. In certain embodiments, the hydrogen-accepting component has an average particle size in the range of about 180 μm to about 425 μm. In many embodiments, the hydrogen-accepting component includes zirconium. In preferred embodiments, the hydrogen-accepting component includes zirconium particles with a particle size ranging from about 180 μm to about 425 μm.

In an embodiment, the method may comprise exposing a Mo/H-ZSM-5 pre-catalyst to a fluid composition containing methane and argon to convert molybdenum oxides of the Mo/H-ZSM-5 pre-catalyst to a carbidic form of molybdenum thereby forming MoCₓ/H-ZSM-5; and mixing the MoCₓ/H-ZSM-5 with zirconium particles to form a catalyst, such as an interpellet physical mixture of Zr and MoCₓ/H-ZSM-5.

In an embodiment, the method of preparing a catalyst and/or pre-catalyst may comprise one or more of the following steps: contacting a first active metal precursor and a porous support sufficient to deposit the first active metal precursor on a surface of the porous support and form a first pre-catalyst; treating the first pre-catalyst sufficient to convert the first active metal precursor to a second active metal precursor on a surface and/or pores of the porous support and form a second pre-catalyst; contacting the second pre-catalyst with a fluid composition containing at least methane sufficient to convert the second active metal precursor to a carbidic form and form a third pre-catalyst, and contacting the third pre-catalyst with a hydrogen accepting component to form a catalyst.

In an embodiment, the method of preparing a catalyst and/or pre-catalyst may comprise one or more of the following steps: heating a zeolite precursor to form a proton form of a zeolite; contacting (grinding and heating) the proton form of the zeolite with a Mo precursor and/or W precursor sufficient for the Mo precursor and/or W precursor to disperse on a surface of the proton form of the zeolite and form a first intermediate; heating the first intermediate sufficient for the Mo precursor and/or W precursor to migrate into pores of the proton form of the zeolite and form a second intermediate (e.g., Mo/H-ZSM-5); optionally reducing a particle size of the second intermediate; exposing the second intermediate to at least methane sufficient for carburization of the Mo precursor and/or W precursor thereby forming a third intermediate; and contacting the third intermediate with a metal to form a catalyst.

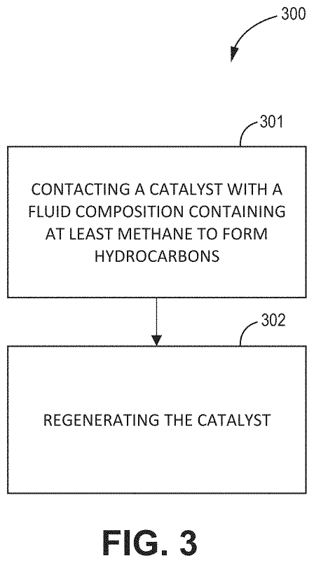

FIG. 3 is a flowchart of a method of forming hydrocarbons, according to one or more embodiments of the present disclosure. As shown in FIG. 3, the method may comprise contacting 301 a catalyst with a fluid composition containing at least methane to form hydrocarbons and optionally regenerating 302 the catalyst. The steps of 301 and 302 may be repeated one or more times. For example, in certain embodiments, the method of forming hydrocarbons is a method of producing aromatics, the method comprising contacting a methane-containing feed stream with a pre-carburized catalyst to form aromatics and hydrogen, wherein the pre-carburized catalyst is disposed within a reactor and comprises a carbidic form of an active metal supported on a proton form of a zeolite, wherein the reactor further comprises a hydrogen-accepting component that removes at least a portion of said hydrogen. In an embodiment, the hydrogen-accepting component is Zr particles or any of the hydrogen-accepting components disclosed herein.

The step 301 includes contacting a catalyst with a fluid composition containing at least methane to form hydrocarbons. The contacting may include bringing the catalyst and the fluid composition into physical contact, or immediate or close proximity. Examples of the contacting may include, but are not limited to, one or more of feeding, flowing, passing, and pumping. The contacting may proceed under conditions suitable for dehydroaromatization of methane and/or non-oxidative methane aromatization to yield hydrocarbons, such as aromatics. For example, the contacting may proceed at or to temperatures ranging from about 500 K to about 1500 K. In many embodiments, the contacting may proceed at or to temperatures ranging from about 900 K to about 1200 K. In preferred embodiments, the contacting may proceed at or to temperatures ranging from about 973 K to about 1193 K. In other embodiments, the contacting may proceed at or to temperatures of less than about 500 K and/or greater than about 1500 K. The contacting may proceed at or to a pressure ranging from about greater than 0 kPa to about 150 kPA. In many embodiments, the contacting may proceed at or to a pressure ranging from about 3 kPa to about 100 kPA, such as about 3.28 kPa to about 95.13 kPa. In some embodiments, the contacting proceeds at or to about atmospheric pressure. In other embodiments, the contacting may proceed at or to a pressure greater than about 150 kPa. In certain embodiments, prior to the contacting, the method includes exposing a pre-catalyst comprising an active metal supported on a proton form of a zeolite to methane to obtain the pre-carburized catalyst.

The catalyst may include any of the catalysts and/or pre-catalysts described herein. For example, in an embodiment, the catalyst may include a pre-catalyst, such as the Mo/H-ZSM-5 pre-catalyst. In an embodiment, the catalyst may include a catalyst including Zr+MoCₓ/H-ZSM-5. In an embodiment, the catalyst includes a zirconium mixed with a porous support, wherein the porous support includes a proton form of a zeolite and a carbidic form of Mo or W deposited on a surface and pores of the proton-form zeolite. The fluid composition may include at least methane. In some embodiments, the fluid composition may include one or more other chemical species. For example, in an embodiment, the fluid composition may include natural gas feedstock.

The contacting may form hydrocarbons, such as aromatics. For example, in many embodiments, the major products may include one or more of benzene, toluene, naphthalene (e.g., with greater than about 92% carbon selectivity), with the minor products including xylenes and C₁₀⁺ (e.g., with less than about 6% carbon selectivity). C₂Hₓ products, such as ethane and ethylene, may also be produced (e.g., accounting for less than about 3% of total products on a carbon basis). In certain embodiments, the hydrocarbons, or aromatics, are formed without an induction period.

While not wishing to be bound to a theory, it is believed that the C—H bonds in methane may be activated on MoCₓ clusters formed during carburization, which may also catalyze dehydrogenation to form C₂Hₓ species which subsequently undergo oligomerization/hydrogen transfer on residual Bronsted acid sites within zeolite channels to form aromatics. In addition, in-situ removal of hydrogen may be achieved using the hydrogen-acceptor component, such as zirconium, resulting in the formation of hydride, for example, ZrHₓ species. The in-situ removal of hydrogen overcomes thermodynamic equilibrium constraints to enhance methane conversion and aromatic product formation. In certain embodiments, the single-pass methane conversion can be greater than the equilibrium methane conversion under the same reaction conditions. For example, single-pass methane conversion may be as high as 27%, as compared to 10% equilibrium conversion.

The step 302 includes regenerating the catalyst. The regenerating may include desorption of species deposited on the catalyst during methane dehydroaromatization. In many embodiments, the catalyst may be regenerated by contacting the catalyst with an inert flow under thermal treatment. For example, the catalyst may be contacted with a helium flow at temperatures ranging from about 900 K to about 1200 K, or preferably about 1193 K. The regenerating may release hydrogen absorbed by the hydrogen-acceptor during methane dehydroaromatization and that resulted in the formation of hydrides, thereby regenerating the hydrogen-acceptor component. In addition or in the alternative, the released hydrogen may hydrogenolyze carbonaceous species deposited during methane dehydroaromatization.

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### Example 1

The following Example relates to the absorptive hydrogen scavenging for enhanced aromatics yield during non-oxidative methane aromatization on Mo/H-ZSM-5 catalysts. More specifically, this Example describes an increase in yields of desired products in non-oxidative methane dehydrogenation on Mo/HZSM-5 formulations by introducing an additional function that scavenges byproduct hydrogen thereby lifting thermodynamic constraints on attainable yields and increasing rates of products formation. The increase in aromatics yield by addition of a hydrogen-adsorbent was rationalized. It was surmised that an optimum in efficacy of hydrogen removal existed because excess hydrogen removal should result in multi-ring aromatic and coke formation as the reversibilities for these reactions depend more sensitively on H₂ concentration than the reversibilities for ethylene and benzene. The inquiry focused on, among other things, the effects of proximity between Mo/ZSM-5 and the hydrogen adsorptive function and what material characteristics of the additive determined its effectiveness as a hydrogen adsorbent.

It was proposed to examine the effects of proximity between Mo/ZSM-5 domains and Zr domains on rates and selectivity in polyfunctional CH₄ dehydroaromatization. Molecular transport events conveyed hydrogen byproduct formed at Mo/ZSM-5 domains to Zr domains which stoichiometrically scavenge hydrogen forming a metal (oxy)hydride. The consequences of the kinetic relevance of this molecular transport event on rates and selectivity was assessed by studying mixtures of Mo/ZSM-5 and Zr in three configurations spanning seven decades of length scales: alternating stratified layers of Mo/ZSM-5 and Zr (10⁻² m) (FIG. 1A), interparticle physical mixtures (10⁻⁴ m) (FIG. 1B), and intraparticle physical mixtures (10⁻⁶ m) (FIG. 1C). It is believed that Mo/ZSM-5 encapsulated Zr particles (10⁻⁹ m) shown in FIG. 1D is to be advantageous.

Inert quartz wool was utilized to separate stratum of Mo/ZSM-5 from stratum of Zr-adsorbents, and homogeneous interparticle and intraparticle physical mixtures were prepared. Zr- and Mo-exchanged ZSM-5 formulations were prepared following protocols described herein. This portfolio of materials, systematically variate in proximity between Mo/ZSM-5 and Zr, was subjected to a suite of structural and chemical characterization protocols to determine bulk structure and morphology (X-ray diffraction and electron microscopy), chemical composition and speciation, (ICP-OES, X-ray absorption and photoelectron spectroscopy), and active site identification, enumeration, and accessibility (transient sorption measurements and temperature programmed surface reactions) of both as-prepared and post-reaction catalysts.

It was expected that the kinetic relevance of hydrogen transport events from the Mo/ZSM-5 domain to the Zr domain would increase with decreasing proximity, and consequently, it was expected that the efficacy of hydrogen removal would decrease with decreasing proximity. It was posited that the selectivity to desired products (e.g., ethylene and benzene) was maximized at an intermediate value of hydrogen removal efficacy. It was anticipated that too great of hydrogen removal efficacy would increase net rates of highly unsaturated species more strongly than ethylene and benzene because the hydrogen concentration dependencies of net rates increased with increasing product unsaturation. Tuning length scales to tune kinetic relevance of hydrogen transport provided one handle for optimizing desired selectivity in polyfunctional catalysis for CH₄ dehydroaromatization. The identity, structure, and composition of the hydrogen removal function was another handle.

Chemical transient studies and Raman spectroscopy evinced formation of (Mo₂O₅)²⁺ dimers on air treatment of MoO₃/H-ZSM-5 physical mixtures at 973 K and removal of 2.44±0.1 O:Mo during initial CH₄ reactions with MoOₓ precursors in a stoichiometric reaction that led to MoCₓ moieties. The resulting MoCₓ/H-ZSM-5 formulation exhibited a steady-state forward benzene synthesis rate of (5.05±0.09)×10⁻⁴ mol mol_{Mo}⁻¹ s⁻¹ during methane dehydroaromatization at 973 K. Addition of Zr metal particles to MoCₓ/ZSM-5 in interpellet mixtures resulted in a 2.7× increase in methane converted and a concurrent 1.4, 2.1, 2.6, and 5.6-fold increase in benzene, naphthalene, toluene, and C₁₀+ yields, respectively, in reference to a conventional MoCₓ/ZSM-5 catalyst at equivalent time-on-stream (8.7 ks). The maximum methane conversion on these interpellet mixtures exceeded ˜27% demonstrating that equilibrium limitations encountered in methane dehydroaromatization on MoCₓ/ZSM-5 were circumvented by addition of Zr as a hydrogen absorbent. Subsequent thermal treatment of the catalyst in helium flow at 973 K resulted in desorption of absorbed hydrogen and in regeneration of the Zr absorbent leading to partial regeneration of the polyfunctional catalyst formulation yielding above equilibrium methane conversions. Hydrogen uptake experiments on Zr metal at 973 K demonstrated that zirconium metal forms ZrH_{1.75} on hydrogen exposure (3.28-95.13 kPa) as reconciled by X-ray diffraction.

### Materials and Methods

Catalyst Synthesis.

NH₄-ZSM-5 (Zeolyst International, Si/Al=11.5, CBV 2314) was converted to H-ZSM-5 by treating in dry air (˜1.67 cm³ s⁻¹) to thermally decompose NH₄⁺ to H⁺ and NH₃ (g) by increasing the temperature from room temperature to 773 K at ˜0.0165 K s⁻¹ and holding at 773 K for ˜36 h. Intimate mixtures containing Mo: Al_{f}˜0.25 were prepared by grinding together MoO₃ (Sigma-Aldrich, 99.9%) and H-ZSM-5 powders in an agate mortar and pestle for ˜0.25 h. The mixture was heated from room temperature to 623 K at ˜0.0167 K s⁻¹ and held at this temperature for 15 h in dry air (˜0.67 cm³ s⁻¹) resulting in water removal and dispersion of MoO₃ on the external surface of the zeolite. Finally, the mixture was heated to 973 K at ˜0.167 K s⁻¹ and held at this temperature for 10 h to facilitate molybdenum oxide migration into the zeolite pores. The catalyst prepared via the above-mentioned protocol is referred to as Mo/H-ZSM-5 in this Example. The Mo/H-ZSM-5 powder was pressed to form pellets that were then crushed and sieved to obtain particle sizes between 180 and 425 μm (mesh 40-80) for use in catalytic reactions. Pure zirconium (Zr) metal was obtained from American Elements (PN # ZR-M-0251M-GR.1T2MM, 99.5+% purity, metal basis) as granule-shaped particles (1-2 mm granule size) which were crushed and sieved to obtain particle sizes between 180 and 425 μm (mesh 40-80) for use in the catalytic reactions.

Catalyst Characterization.

The bulk structure of the zirconium (American Elements PN # ZR-M-0251M-GR.1T2MM, 99.5+% purity, metal basis) and zirconium hydride particles (prepared as discussed in Section 2.4) was determined by X-ray diffraction (XRD) using a Bruker D8 Discover 2D X-ray diffractometer with a 2-D VÅNTEC-500 detector, Co K_{α} X-ray radiation with a graphite monochromator, and a 0.8 mm point collimator. Scans were measured in three measurement frames at 20° (2θ), 45° (2θ), and 70° (2θ) at 900 s frame⁻¹ while rotating the sample. Area detector images were finally converted to one-dimensional intensity vs. 2θ data sets by using an averaging integration algorithm and the radiation wavelength was recalibrated for Cu K_{α} wavelength (λ=1.541 Å).

Raman spectra of three samples—a physical mixture of MoO₃ and H-ZSM-5 with Mo:Al_{f}˜0.25, Mo/H-ZSM-5 with Mo:Al_{f}˜0.25, and H-ZSM-5 air treated at 973 K for 5 h—were collected on a WITec alpha 300R confocal Raman microscope (WITec Instrument Corp., Germany) equipped with a frequency doubled 532 nm Nd:YAG laser, a 100× Nikon air objective with a numeric aperture of 0.90, a UTS300 spectrometer with 1800 groves/mm grating, and a DV401 CCD detector. The lateral spatial resolution was ˜0.5 μm and the data were processed using the WITec Project 4.05 software.

Brønsted acid site density of the H-ZSM-5 sample employed was measured using NH₃ temperature-programmed-desorption. H-ZSM-5 (˜0.160 g) was treated in flowing NH₃ (1.67 cm³ s⁻¹, 1.01% NH₃ in He, Praxair, certified standard) at 423 K for ˜1.5 ks, purged in flowing He (Minneapolis Oxygen, 99.997%; 1.67 cm³ s⁻¹) for ˜28.8 ks, and ramped to 823 K at 0.167 K s⁻¹ in flowing He and Ar (Matheson, UHP/Zero; used as an internal standard) while continuously monitoring the effluent via an online mass spectrometer (MKS Cirrus, m/z=16, 17, 18, 40) to quantify desorbed NH₃. The H⁺ site density was calculated by assuming unit stoichiometry between H⁺ and NH₃ desorbed from 423 to 823 K.

Methane Dehydroaromatization Catalytic Reactions.

Methane dehydroaromatization reactions were performed in a fixed bed tubular quartz reactor (I.D. 10 mm) with an outer thermowell above the porous quartz frit (for holding the catalyst bed stationary) to hold a thermocouple to monitor the reaction temperature. The catalyst sample was heated in helium flow (˜0.33 cm³ s⁻¹, UHP, Minneapolis Oxygen) from room temperature to the reaction temperature, 973 K, at ˜0.18 K s⁻¹ in a resistively heated furnace (National Element FA120). Axial temperature gradients in the reactor were minimized by using an annular inconel cylinder to fill the vacant space in the furnace. A feed gas mixture of CH₄/Ar (90 vol % CH₄ and 10 vol % Ar, total feed flow rate ˜0.27 cm³ s⁻¹, UHP, Matheson Tri-Gas) was introduced to the reactor to perform methane reactions at 973 K and atmospheric pressure, with Ar serving as an internal standard. All flow lines were heated to temperatures in excess of ˜473 K via resistive heating to prevent condensation of effluents. The composition of the reactor effluent was analyzed using a mass spectrometer (MKS Cirrus 200 Quadrupole MS system) and a gas chromatograph (Agilent 7890) equipped with a methyl-siloxane capillary column (HP-1, 50 m×320 μm×0.52 μm) connected to a flame ionization detector (FID) for detection of hydrocarbons and a GS-GasPro column (60 m×0.320 mm) connected to a thermal conductivity detector (TCD) for detection of permanent gases (H₂, Ar, and CH₄). Transient product evolution throughout the course of the reaction was measured with an online mass spectrometer (MS) (MKS Cirrus 200 Quadrupole MS system) connected to the outlet of the GC. The number of removed O atoms during carburization of molybdenum oxide was determined by the cumulative amount of CO and CO₂ eluted as calculated from the transient MS signal with Ar as an internal standard.

The samples designated as MoCₓ/H-ZSM-5 in this Example were prepared by exposing the Mo/H-ZSM-5 formulation to a CH₄/Ar mixture (90 vol % CH₄ and 10 vol % Ar, total feed flow rate ˜0.27 cm³ s⁻¹) for ˜15.5 ks (for catalyst loading ˜1.2 g) leading to complete carburization of the molybdenum oxide and subsequently, switching the feed to helium (˜0.83 cm³ s⁻¹). The sample was flushed in helium flow (˜0.83 cm³ s⁻¹) at 973 K for ˜0.9 ks and then cooled to room temperature in the same helium flow. For the formulation designated as Zr+MoCₓ/H-ZSM-5 in this Example, the MoCₓ/H-ZSM-5 sample (˜1.2 g) was unloaded from the reactor and mixed with Zr particles (180-425 μm, ˜2.4 g) in a glove bag (Sigma Aldrich, Z530212, AtmosBag, two-hand, non-sterile, size M, Zipper-lock closure type) under inert (helium) atmosphere to prevent oxidation of the carburized catalyst. This procedure gave interpellet physical mixtures of Zr and MoCₓ/H-ZSM-5. The interpellet mixture was subsequently loaded in the same quartz reactor under an inert atmosphere and transferred to the furnace without exposing the mixture to ambient conditions. The mixture was heated to the reaction temperature, ˜973 K, in helium flow (˜0.33 cm³ s⁻¹) at ˜0.18 K s⁻¹ before performing methane dehydroaromatization reactions.

Experiments to demonstrate in-situ regeneration of the absorbent function involved running methane dehydroaromatization on Zr+MoCₓ/H-ZSM-5 formulations for ˜3.6 ks. Subsequently, the flow was switched to helium (˜0.83 cm³ s⁻¹) for ˜61.2-82.8 ks and hydrogen in the reactor effluent was monitored using a mass spectrometer before performing methane dehydroaromatization reactions following the procedure described above.

Hydrogen Uptake on Zirconium.

The hydrogen uptake capacity of Zr was examined by loading ˜2.4 g of Zr particles (180-425 μm) in a tubular quartz reactor (I.D. 10 mm) and heating the sample in helium flow (˜0.83 cm³ s⁻¹) to ˜973 K from ambient temperature at ˜0.18 K s⁻¹. Subsequently, the flow was switched to a H₂/Ar gas mixture (3.28-95.13 kPa H₂/balance Ar, total flow rate ˜1.7 cm³ s⁻¹) and the reactor effluent transient was monitored via a mass spectrometer. Once the hydrogen (m/z=2) mass spectrometer signal reached a steady value in ˜0.4-12 ks depending on the feed hydrogen pressure, the feed was switched to helium flow (˜0.83 cm³ s⁻¹).

Temperature-Programmed Desorption (TPD).

Temperature-programmed desorption in helium flow was performed either post methane dehydroaromatization on Zr+MoCₓ/H-ZSM-5 or after hydrogen uptake on Zr particles. The feed was switched from a CH₄/Ar or a H₂/Ar mixture to helium (˜0.83 cm³ s⁻¹) at 973 K. The sample was flushed in helium flow at ˜973 K for ˜0.9 ks. Subsequently, the sample was heated to ˜1193 K at ˜0.061 K s⁻¹ and held at ˜1193 K for 25-35 ks in helium flow. The reactor effluent was monitored using a mass spectrometer to quantify hydrogen and methane removed from the catalyst sample during TPD with helium as an internal standard. Finally, the catalyst (Zr+MoCₓ/H-ZSM-5) was cooled to room temperature whereas Zr particles were cooled to ˜973 K. Subsequent hydrogen uptake/TPD cycles at different hydrogen pressures (3.28-95.13 kPa H₂/balance Ar, total flow rate ˜1.7 cm³ s⁻¹) were performed on a single Zr sample (˜2.4 g loading) as discussed in Section 3.4.

### Results and Discussion

The addition of Zr to pre-carburized Mo/H-ZSM-5 formulations (denoted as MoCₓ/H-ZSM-5 in this Example) resulted in formation of ZrHₓ species during methane dehydroaromatization reactions at 973 K resulting in ˜27% maximum single-pass methane conversion and an increase in yield of methane-derived hydrocarbon products at equivalent time-on-stream because equilibrium limitations were transiently circumvented by the hydrogen-absorptive Zr function. Thermal treatment of the Zr+MoCₓ/H-ZSM-5 formulation in helium flow resulted in regeneration of the Zr-absorbent leading to above-equilibrium methane conversions in successive reaction-regeneration cycles.

Chemical and Structural Characterization of Mo/H-ZSM-5 Formulations.

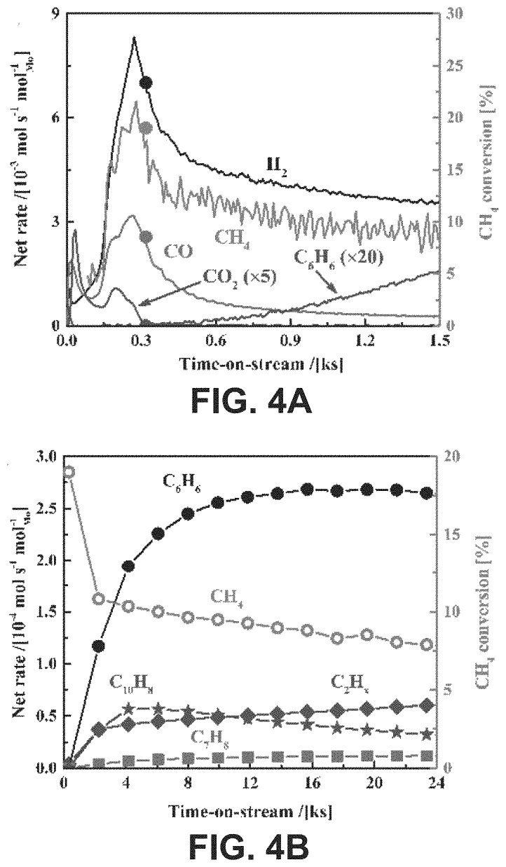

The transient evolution of products during the induction period of methane dehydroaromatization (DHA) on Mo/H-ZSM-5 catalyst at 973 K is shown in FIGS. 4A-4B. CO, CO₂, and H₂ were observed as the dominant products before the evolution of any hydrocarbon products (as shown in FIG. 4A). The formation of CO₂ attained a maximum at ˜0.2 ks whereas CO and H₂ formation rates were maximized at ˜0.3 ks, concurrent with negligible CO₂ formation. Exposure of Mo/H-ZSM-5 to methane removed ˜2.44±0.1 oxygen atoms per Mo during carburization dynamics of ˜15.5 ks as assessed by measuring the cumulative amount of oxygen-containing products (CO and CO₂) in the reactor effluent during this time interval. These observations elucidated the stoichiometric reaction of MoOₓ precursors with methane to form MoCₓ clusters and suggested that (Mo₂O₅)²⁺ dimers occupying two proximate Al site were formed on air treatment of MoO₃ and H-ZSM-5 physical mixtures at 973 K, consistent with earlier reports. An induction period of ˜0.6 ks was observed for the formation of benzene pertaining to the time needed for O removal prior to aromatics formation. As shown in FIG. 4B, C₂Hₓ (ethane and ethylene) products were formed immediately on exposure of Mo/H-ZSM-5 to methane at 973 K and the rate gradually increased to a steady state value of ˜6×10⁻⁵ mol s⁻¹ mol_{Mo}⁻¹. In contrast, the primary product of methane DHA, benzene, was evolved initially at ˜0.6 ks and increased monotonically to a maximum formation rate of ˜2.7×10⁻⁴ mol s⁻¹ mol_{Mo}⁻¹ at ˜15.5 ks after complete carburization of (Mo₂O₅)²⁺ dimers to MoCₓ.

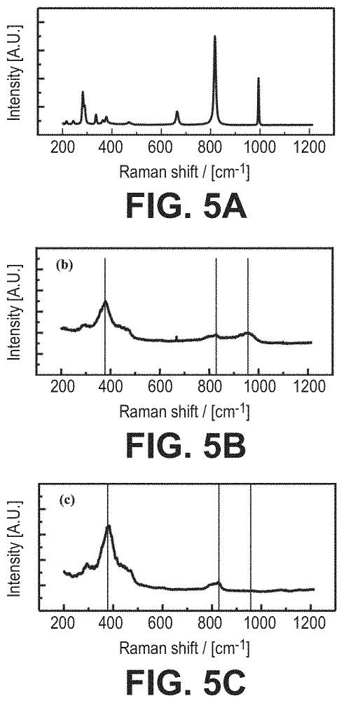

FIGS. 5A-5C show the Raman spectra of three samples: a physical mixture of MoO₃ and H-ZSM-5 with Mo:Al_{f}˜0.25, Mo/H-ZSM-5 with Mo:Al_{f}˜0.25, and H-ZSM-5 air treated at 973 K for 5 h. The MoO₃ and H-ZSM-5 physical mixture (FIG. 5A) showed Raman bands at 820, 996, and 300 cm⁻¹ attributed to the antisymmetric stretching mode of Mo—O—Mo, the stretching mode of Mo═O, and the bending mode of Mo═O bonds in bulk MoO₃ crystallites, respectively. Mo/H-ZSM-5 (FIG. 5B) showed much weaker bands at 820 and 996 cm⁻¹ indicating that bulk MoO₃ crystallites dispersed during thermal treatment at 973 K. Mo/H-ZSM-5 also showed weak bands at 376 and 820 cm⁻¹ corresponding to framework vibrations as affirmed by the presence of these bands in Raman spectra for a H-ZSM-5 sample air treated at 973 K for 5 h (FIG. 5C). These data also suggested that crystallinity of the zeolite framework was preserved during thermal treatment of Mo/HZSM-5 formulations. A new band at 970 cm⁻¹ was observed for Mo/H-ZSM-5 (FIG. 5B) which was assigned by Li et al. to one of the stretching modes of Mo═O bonds in (Mo₂O₅)²⁺ dimers formed within the H-ZSM-5 channels after MoOₓ thermal exchange at 973 K. The appearance of a new 970 cm⁻¹ band and the disappearance of bands corresponding to bulk MoO₃ in Raman spectra of Mo/H-ZSM-5 were consistent with earlier reports, further supporting (Mo₂O₅)²⁺ dimer formation in Mo/H-ZSM-5.

Formation of (Mo₂O₅)²⁺ dimers upon ion exchange of MoO₃ with two Al proximate centers within H-ZSM-5 channels was evidenced using X-ray absorption spectroscopy and supported by computational chemistry studies. Mo/H-ZSM-5 showed a pre-edge feature in near-edge X-ray absorption spectra similar to MgMo₂O₇ which contained ditetrahedral Mo centers and also a post-edge energy (20 keV) absorbance similar to MgMo₂O₇ as opposed to MoO₃ which contained distorted octahedral Mo centers. In order for (Mo₂O₅)²⁺ moieties to stabilize, the framework oxygen connected to Al atoms should reside within 0.42-0.55 nm of each other. Computational studies in other reports show that the fraction of Al atoms that reside within 0.55 nm of another framework Al for a Si:Al_{f}˜15 is ˜0.42 assuming random Al occupancy of T sites in the sample. It has also been demonstrated that at Mo loadings in excess of 2 wt % for H-ZSM-5 with Si:Al˜13, irreversible damage to the zeolite framework occurred due to MoOₓ reaction with framework Al leading to aluminum molybdate formation which was inactive for methane DHA. An upper limit to Mo loading in H-ZSM-5 catalysts for methane DHA applications was attributed to the unavailability of enough Al pairs to accommodate all Mo dimers which led to the need for migration of Al atoms during thermal treatment leading to dealumination. Consequently, a Mo:Al_{f}˜0.25 for a H-ZSM-5 catalyst with Si:Al˜11.5 was used to ensure the required number of Al atom pairs were available for (Mo₂O₅)²⁺ dimer formation and stabilization within zeolite channels. The concentration of free Brønsted acid sites in H-ZSM-5 (Si/Al=11.5) was determined via NH₃ uptake to be 1.21×10⁻³ mol g_{cat}⁻¹, which was similar to the concentration of Al in the zeolite as calculated from the Si-to-Al ratio (1.33×10⁻³ mol g_{cat}⁻¹).

CH₄ Dehydroaromatization on Mo/H-ZSM-5 and MoCₓ/H-ZSM-5 Catalysts.

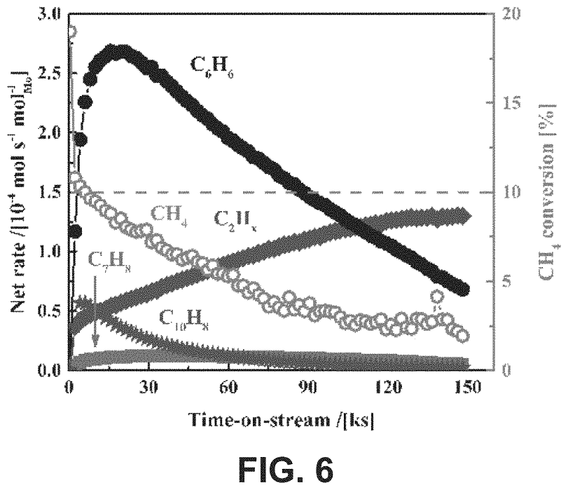

FIG. 6 shows methane conversion and net product formation rates as a function of time-on-stream for Mo/H-ZSM-5 at 973 K. Near equilibrium conversion (˜10%) was observed after carburization was complete at ˜15.5 ks. Methane conversion monotonically decreased at long times-on-stream (exceeding ˜26 ks). C₂Hₓ (ethane and ethylene) was formed during the induction period and its formation rate steadily increased with time-on-stream. Benzene, toluene, and naphthalene were the major aromatic products formed after an induction period during methane dehydroaromatization on Mo/H-ZSM-5 at 973 K. Benzene and toluene net formation rates increased with time-on-stream to achieve maxima of 2.70×10⁻⁴ mol s⁻¹ mol_{Mo}⁻¹ and 1.21×10⁻⁵ mol s⁻¹ mol_{Mo}⁻¹, respectively and subsequently, monotonically decreased at longer times-on-stream. These net benzene formation rates were similar to those reported by others, which included (1.7×10⁻⁴ mol s⁻¹ mol_{Mo}⁻¹), (1.4×10⁻⁴ mol s⁻¹ mol_{Mo}⁻¹), (2.9×10⁻⁴ mol s⁻¹ mol_{Mo}⁻¹), (4.5×10⁻⁴ mol s⁻¹ mol_{Mo}⁻¹), and (5.0×10⁻⁴ mol s⁻¹ mol_{Mo}⁻¹) for methane DHA on Mo/H-ZSM-5 at 950-973 K. Naphthalene net formation rates also followed a similar trend with rates decreasing at long times-on-stream after reaching a maximum of 5.67×10⁻⁵ mol s⁻¹ mol_{Mo}⁻¹.

Catalyst deactivation with time-on-stream was attributed to the continuous buildup of coke within the zeolite channels which presumably led to reduction of effective zeolite diameter, thus inhibiting the formation of bulky aromatics like naphthalene. DME titrations, XPS, ion-scattering spectroscopy, and FT-IR measurements by others demonstrated a reduction in the number of acid sites as well as formation of carbonaceous deposits in zeolite channels at long times-on-stream during methane DHA on Mo/H-ZSM-5. It was observed that benzene and naphthalene selectivity to be ˜67% and ˜21% respectively at ˜15 ks time-on-stream which were typical for methane DHA reactions on Mo/H-ZSM-5. Aromatic selectivity (naphthalene and benzene) decreased at longer times-on-stream with a concurrent increase in C₂Hₓ selectivity, consistent with deactivation of acidic sites in zeolites by coke deposition which limited access to these oligomerization/dehydroaromatization sites.

The induction (carburization) period and the initial increase and subsequent reduction in aromatic formation rates with time-on-stream was suggestive of a bifunctional catalytic mechanism wherein C—H bonds in CH₄ were activated on MoCₓ clusters formed during carburization which also catalyzed dehydrogenation to form C₂Hₓ species which subsequently underwent oligomerization/hydrogen transfer on residual Brensted acid sites within zeolite channels to form aromatics (benzene and naphthalene). Others have demonstrated that bulk molybdenum carbide catalyzed methane conversion to C₂ products but did not yield any aromatics. Furthermore, others also showed that unsupported Mo₂C catalyzed ethane dehydrogenation to ethylene but not aromatics whereas MoCₓ/H-ZSM-5 catalyzed ethane conversion to benzene. Formation of MoCₓ species in zeolite micropores and their subsequent function as catalytic centers for methane DHA has been established through numerous characterization studies including Raman spectroscopy, X-ray absorption, X-ray photoelectron spectroscopy, ion sputtering spectroscopy, UV-visible near infrared spectroscopy, and ⁹⁵Mo nuclear magnetic resonance (NMR). One report demonstrated, using DME chemical titrations during methane DHA on Mo/H-ZSM-5 at 950 K, that the concentration of free Brønsted acid sites increased with carburization time due to MoCₓ cluster formation from (Mo₂O₅)²⁺ species which had exchanged with the Brønsted acid sites during Mo/H-ZSM-5 preparation. Subsequently, the number of DME accessible protons decreased with time-on-stream which was attributed to adsorption of aromatics formed on regenerated acid sites, as suggested by a concurrent increase in benzene formation rate. These characterization studies were in line with the bifunctional mechanism for methane DHA on Mo/H-ZSM-5 catalyst at 973 K.

The exact stoichiometry and coordination of MoCₓ clusters are still debated in the literature, with emphasis on the role of Mo speciation on methane DHA reactions, but the reduced carbidic nature of active Mo-centers and their proficiency during methane DHA reactions is well established. All rates reported in this Example were normalized to the total number of Mo atoms to account for all available Mo atoms resulting in the lowest possible rate.

The forward rate of benzene formation for the stoichiometric reaction of CH₄ to H₂ and C₆H₆ (equation 1) on Mo/H-ZSM-5 at 973 K was calculated by analyzing the net rate of benzene formation and effluent hydrogen, benzene, and methane pressures in the regime (12-22 ks time-on-stream) in which net benzene formation rate was invariant.

CH₄↔⅙C₆H₆+ 3/2H₂ (1)

The approach to equilibrium for the reaction in equation 1, η, was calculated via equation 2 using outlet pressures of benzene, hydrogen, and methane and the equilibrium constant determined from thermodynamic values at 973 K (K_{eq}=0.0302). The forward rate of benzene formation (R_{for}) was related to the net benzene rate (Rₙₑₜ) and η as shown in equation 3. Using the calculated values of Rₙₑₜ (2.7×10⁻⁴ mol s⁻¹ mol_{Mo}⁻¹) and η(˜0.5), the forward rate of benzene formation (R_{for}) was (5.05±0.09)×10⁻⁴ mol s⁻¹ mol_{Mo}⁻¹. This forward rate of benzene formation was similar to rates reported by others (4.7±0.8×10⁻⁴ mol s⁻¹ mol_{Mo}⁻¹) and (5.03×10⁻⁴ mol s⁻¹ mol_{Mo}⁻¹) for methane DHA on Mo/H-ZSM-5 at 950 K.

$$\begin{matrix}η = \frac{{P}_{{C}_{6} {H}_{6}}^{\frac{1}{6}} {P}_{{H}_{2}}^{\frac{3}{2}}}{{P}_{{CH}_{4}} {K}_{eq}} & ( 2 ) \\ {R}_{for} = \frac{{R}_{net}}{( 1 - η )} & ( 3 )\end{matrix}$$

Another report showed that the net rate of benzene formation systematically decreased with increasing H₂ co-feed concentration (H₂:CH₄=0.09-0.27 molar ratio) and that benzene synthesis rates recover to their pre-H₂ co-feed values upon removal of H₂ from the reactor influent demonstrating that hydrogen did not cause any irreversible structural or chemical modification to the MoCₓ moieties present in the catalyst during methane DHA at 950 K. Moreover, an invariant forward rate of benzene formation (˜(3.8±0.5)×10⁻⁴ mol s⁻¹ mol_{Mo}⁻¹) was obtained for different catalyst loadings (0.1-1.0 g) and H₂:CH₄ co-feeds (0.03-0.11 molar ratio) at 950 K demonstrating that hydrogen did not have any kinetic effect on the rate-limiting step of CH₄ DHA and that hydrogen reduced the aromatic formation rate by increasing the thermodynamic reversibility of methane to benzene reaction. These results showed that methane DHA on Mo/H-ZSM-5 was limited by thermodynamic equilibrium due to inhibition by abundant amounts of hydrogen produced in the catalyst bed.

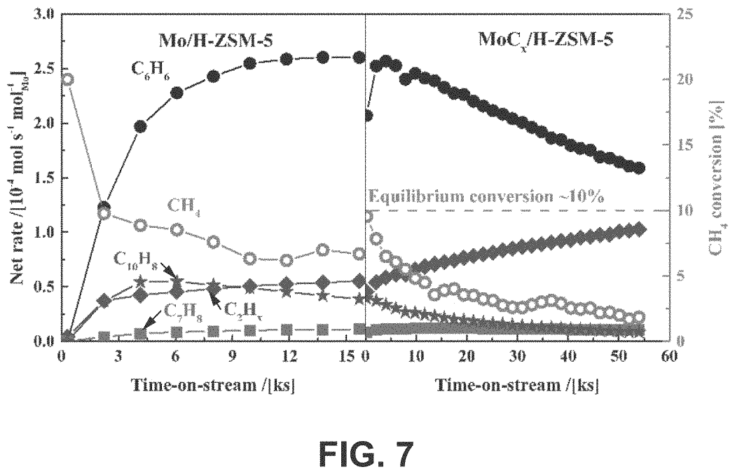

CH₄ reaction on Mo/H-ZSM-5 was performed at 973 K for ˜15.5 ks, during which (Mo₂O₅)²⁺ dimers were carburized to MoCₓ species with O_{removed}:Mo˜2.44±0.1 as CO and CO₂ as noted in Table 1, and then switched the reactor influent to an inert (helium, ˜0.83 cm³ s⁻¹). Methane conversion and product formation rates exhibited a time-on-stream profile similar to Mo/H-ZSM-5 (FIG. 4B) as shown in FIG. 7 (catalyst labeled as Mo/H-ZSM-5). Subsequently, methane DHA was performed on this carburized catalyst at ˜973 K and methane conversion and product rates as a function of time-on-stream are shown in FIG. 7 (catalyst labeled as MoCₓ/H-ZSM-5). As shown in FIG. 7, the induction period in aromatics (benzene and naphthalene) formation observed in the case of Mo/H-ZSM-5 was absent on MoCₓ/H-ZSM-5 due to the presence of catalytically active MoCₓ moieties from the pre-carburization of Mo/H-ZSM-5. The MoCₓ/H-ZSM-5 formulation showed characteristics of Mo/H-ZSM-5 for methane DHA after complete carburization of Mo-oxo species including (i)<10% methane conversion (equilibrium conversion for 6CH₄+↔C₆H₆+9H₂ at 973 K) that decreased with time-on-stream, (ii) benzene, toluene, and naphthalene as the major products with C₂Hₓ formed at <3% carbon selectivity, (iii) steady benzene formation rate for ˜6 ks that subsequently declined with time-on-stream, and (iv) a shift in product selectivity towards C₂Hₓ at longer times-on-stream, as shown in FIG. 7. The forward rate of benzene formation (R_{for}) was ˜(4.92±0.06)×10⁻⁴ mol s⁻¹ mol_{Mo}⁻¹. These observations demonstrated that induction periods corresponding to carburization dynamics were avoided when initiating methane DHA with pre-carburized catalyst formulations and therefore, pre-carburized formulations allowed for assessment of the effects of a hydrogen-absorbent function on mitigating thermodynamic equilibrium limitations in methane DHA as discussed below.

CH₄ Dehydroaromatization on Physical Mixtures of Zr and MoCJH-ZSM-5.

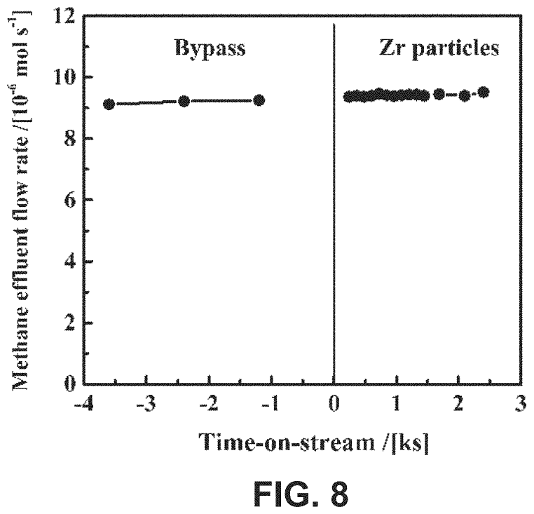

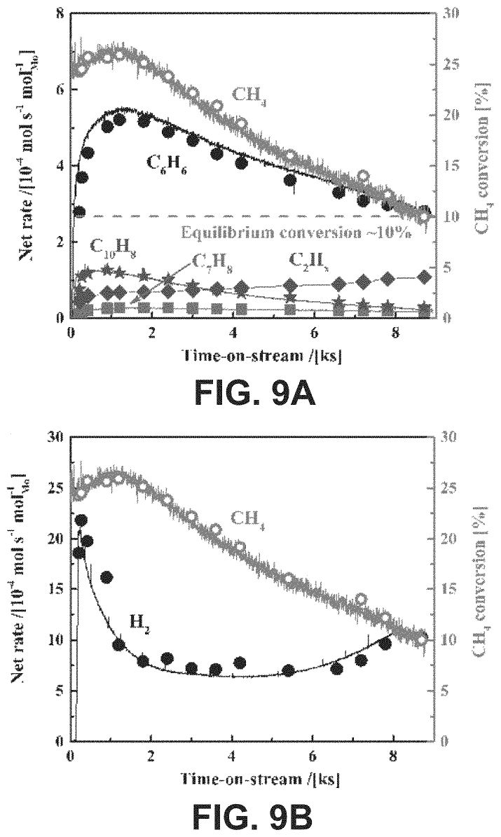

Methane dehydroaromatization was investigated at 973 K on an interpellet physical mixture of zirconium metal and MoCₓ/H-ZSM-5. The physical mixing of Zr particles with the MoCₓ/ZSM-5 formulation was performed in an inert environment to avoid oxidation of MoCₓ species on atmospheric exposure due to their oxophilic nature. Methane flow over Zr particles at ˜973 K did not result in any product (aromatic or C₂Hₓ) formation as shown in FIG. 8. Activity of zirconium metal for methane conversion was tested by flowing methane (total flow rate about 0.21 cm³ s⁻¹, 90 vol % CH₄/balance Ar) over Zr metal particles (about 2.4 g Zr metal) at about 973 K. The time-on-stream evolution of methane effluent flow rate is shown in FIG. 8. Invariant methane flow rate through bypass and Zr particles demonstrated that Zr did not convert methane to any products under reaction conditions employed in this Example. The transient methane conversion and product formation rates for the Zr and MoCₓ/H-ZSM-5 interpellet mixture (2:1 weight ratio) at 973 K as monitored simultaneously using a gas chromatograph and an online mass spectrometer are shown in FIGS. 9A-9B. Addition of hydrogen-absorbing Zr metal to MoCₓ/H-ZSM-5 methane dehydroaromatization catalyst (2:1 weight ratio) resulted in single-pass methane conversion as high as ˜27% due to lifting of thermodynamic equilibrium constraints (˜10% equilibrium conversion). Benzene, toluene, and naphthalene were the major aromatic products observed (>92% carbon selectivity) with minor amounts of xylenes and C₁₀⁺ also formed (<6% carbon selectivity). C₂Hₓ products (ethane and ethylene) were also observed but accounted for <3% of the total products on a carbon basis. As shown in FIG. 9A, methane conversion, benzene, toluene, and naphthalene formation rates increased monotonically to ˜27% at ˜1.2 ks, ˜5.21×10⁻⁴ mol s⁻¹ mol_{Mo}⁻¹ at ˜1.2 ks, ˜2.84×10⁻⁵ mol s⁻¹ mol_{Mo}⁻¹ at ˜1.8 ks, and ˜1.27×10⁻⁴ mol s⁻¹ mol_{Mo}⁻¹ at ˜0.5 ks, respectively, before decreasing with time-on-stream accompanied by a concomitant increase in C₂Hₓ formation rate. C₂Hₓ formation rates increased continuously to ˜1.09×10⁻⁴ mol s⁻¹ mol_{Mo}⁻¹ at ˜9 ks. The effluent hydrogen flow rate during methane reaction normalized by total Mo atoms in the catalyst is shown as a function of time-on-stream in FIG. 9B along with the methane conversion. After an initial increase for ˜0.27 ks in hydrogen flow rate, there was a monotonic decrease in hydrogen eluted for ˜5.1 ks. This decrease in hydrogen elution, despite high methane conversion and aromatic product formation, was attributed to in-situ absorptive hydrogen removal by zirconium particles resulting in hydride formation. After ˜5.1 ks, an increase in the instantaneous hydrogen rate in the reactor effluent was observed despite the continuing decrease in methane conversion and aromatic formation. This increase was presumably due to the saturation of zirconium particles with hydrogen and a concomitant reduction in hydrogen-removal sites (Zr metal particles) in the catalyst bed.

Benzene and naphthalene instantaneous selectivity calculated on a carbon basis were ˜60% and ˜30% at ˜0.3 ks whereas toluene and C₂Hₓ accounted for ˜3% and ˜2%, respectively, of the total carbon in the products observed. The identity and sequence of appearance of products remain unchanged for MoCₓ/H-ZSM-5 and Zr+MoCₓ/H-ZSM-5 suggesting that the bifunctional reaction pathways of methane dehydroaromatization were unperturbed upon Zr addition. The observed enhancement in methane conversion and aromatic product rates can be explained as a consequence of in-situ hydrogen removal resulting in alleviation of thermodynamic equilibrium constraints and was evident when comparing the net formation rate of products and product yields during methane DHA on MoCₓ/H-ZSM-5 and Zr+MoCₓ/H-ZSM-5 at 973 K.

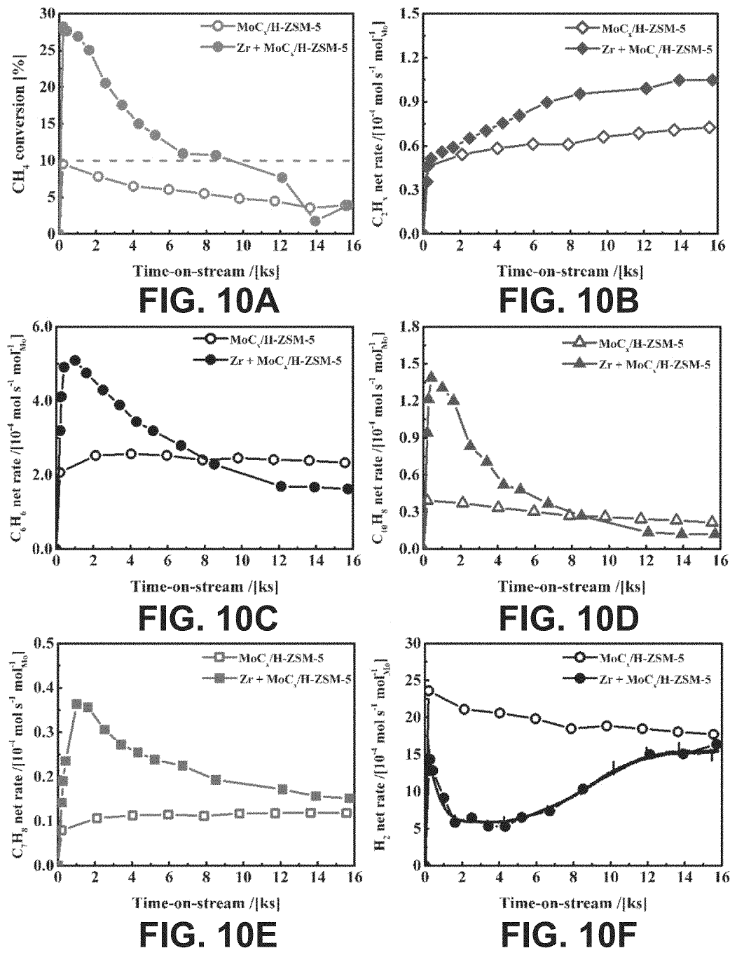

FIGS. 10A-10F shows the comparison of methane conversion, instantaneous product net rate (benzene, naphthalene, toluene, C₂Hₓ), and hydrogen effluent rate as a function of time-on-stream between MoCₓ/H-ZSM-5 and an interpellet physical mixture of Zr and MoCₓ/H-ZSM-5 at similar catalyst loadings (˜1.2 g Mo/H-ZSM-5) during methane DHA at 973 K. Methane conversion as well as aromatic product rates were enhanced while hydrogen effluent rate was suppressed upon Zr addition to MoCₓ/H-ZSM-5 plausibly due to in-situ hydrogen absorptive removal by zirconium (see Section 3.4.1). As shown in FIGS. 10A-10F, the catalyst deactivation evident from the decrease in methane conversion and product rates with time-on-stream for Zr+MoCₓ/H-ZSM-5 was rapid as compared to MoCₓ/H-ZSM-5. The deactivation for Zr+MoCₓ/H-ZSM-5 catalyst formulation can be a consequence of two factors: (i) spatial gradients in hydrogen-absorbing sites across the reactor length due to the gradual stoichiometric reduction of absorbent Zr to form a hydride and (ii) an increase in the rate of formation of unsaturated carbonaceous deposits in zeolite channels due to hydrogen removal leading to a reduction in available Brønsted acid sites for chain growth reactions. The results to-date do not permit distinguishing between these two scenarios.

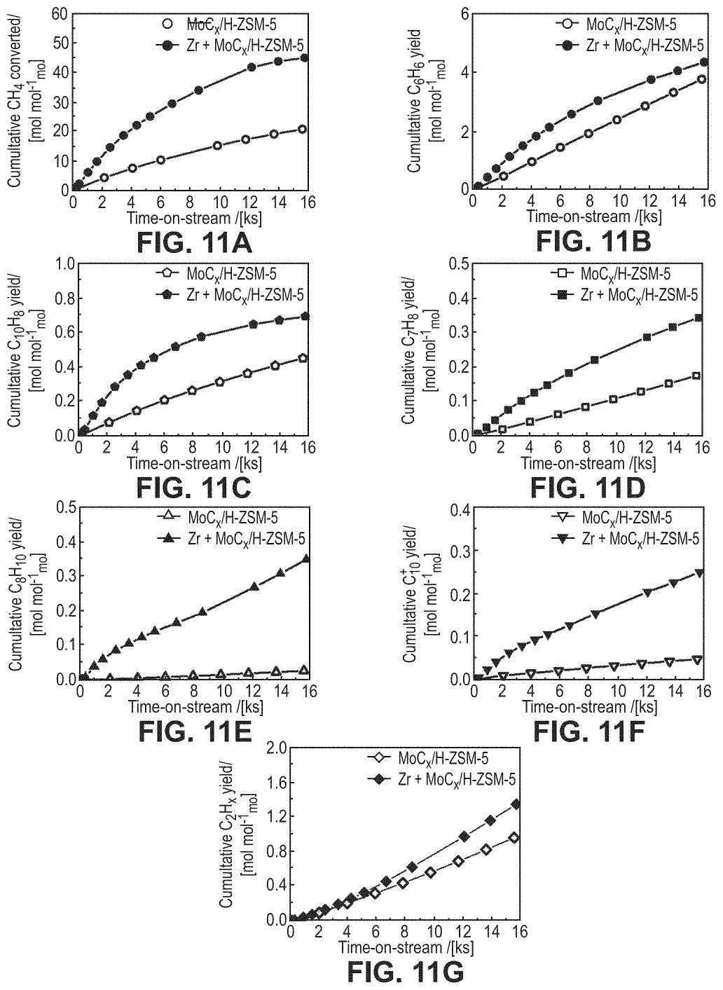

Cumulative product yield for methane DHA on MoCₓ/H-ZSM-5 and Zr+MoCₓ/H-ZSM-5 at time-on-stream, t, was defined as the total number of moles of product formed per total Mo atom in the catalyst at the end of time, t, and was calculated from the area under the curve of the plot for net rate as a function time-on-stream shown in FIGS. 10A-10F. The cumulative yields for aromatic products and the cumulative methane converted per Mo atom (methane turnover) for methane DHA at 973 K on MoCₓ/H-ZSM-5 and on an interpellet physical mixture of Zr and MoCₓ/H-ZSM-5 are shown and compared in FIGS. 11A-11G and Table 1. Zr addition led to enhanced aromatic yields (benzene, naphthalene, toluene, xylenes, C₁₀⁺, C₂Hₓ) and methane turnovers as shown in FIGS. 11A-11G. A comparison of the cumulative yields at 8.7 ks time-on-stream is noted in Table 1. A 2.6-fold increase in methane converted with a concurrent 1.4, 1.6, 2.1, 2.1, and 5.4-fold increase in C₂Hₓ, benzene, naphthalene, toluene, and C₁₀⁺ yields respectively (after 8.7 ks time-on-stream) were achieved via interpellet mixtures of Zr and MoCₓ/ZSM-5 as compared to the conventional MoCₓ/ZSM-5 catalyst. Concurrently, a 0.84× decrease in hydrogen yield in the reactor effluent was observed for Zr+MoCₓ/ZSM-5 evidencing that Zr absorbs hydrogen (as discussed in Section 3.4) formed during methane DHA. Accounting for the total carbon in the products formed using equation 4 below, it was calculated that ˜3.46 C per Mo atom was deposited during methane DHA which is ˜9.6 mol % of the total methane converted (˜35.85 mol_{CH4} mol_{Mo}⁻¹) with Zr addition as compared to ˜0.2 mol % in absence of Zr (total methane converted ˜13.68 mol_{CH4} mol_{Mo}⁻¹), after 8.7 ks time-on-stream evincing the high efficiency of this polyfunctional catalyst formulation to convert methane to aromatic products.

C_{product}=(2×C₂Hₓ+6×C₆H₆+7×C₇H₈+8×C₈H₁₀+10×C₁₀H₈+11×C₁₀⁺) (4)

Hydrogen Uptake and Structural Characterization of Zirconium Metal (Zr) —Hydrogen Uptake of Zirconium Metal.

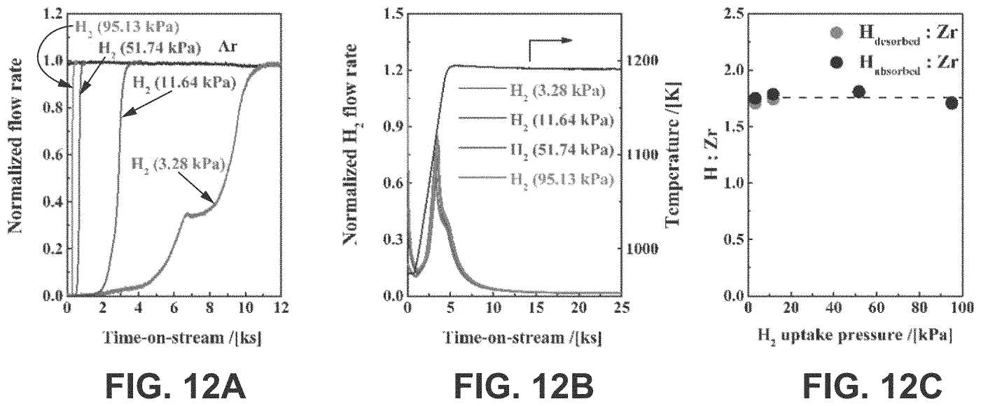

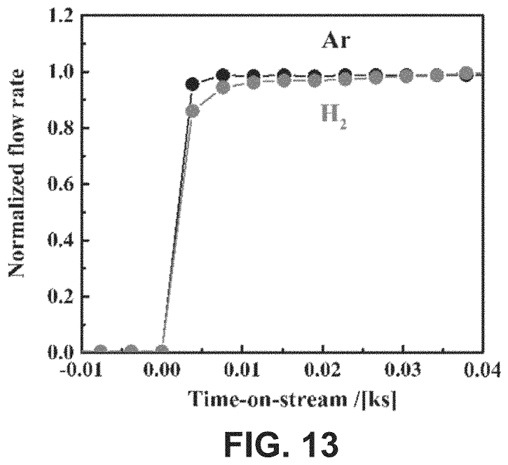

Hydrogen uptake experiments were performed on zirconium metal particles to determine the hydrogen absorption capacity of pure Zr metal at 973 K. FIGS. 12A-12C shows the normalized effluent flow rates, acquired using an online mass spectrometer, for hydrogen and argon during the hydrogen uptake experiment where argon was used as an inert tracer and internal standard. Transient evolution of hydrogen and argon flow, as monitored using an online mass spectrometer, through a blank reactor (total feed flow rate about 1.7 cm³ s⁻¹, H₂/Ar about 95.13 kPa/balance, temperature about 973 K) is shown in FIG. 13. The synchronic breakthrough curve for Ar and H₂ effluent flow rates indicated negligible thermodynamic delay between the two gases in absence of a catalyst. A blank reactor gave a synchronic breakthrough curve for both Ar and H₂ effluent flow rates (FIG. 13) whereas a delay was observed in the breakthrough time for hydrogen as compared to the inert tracer argon when fed through a Zr metal bed (˜2.4 g) as shown in FIG. 12A. The residence time of hydrogen in the reactor can be attributed to hydrogen absorption by Zr metal particles at ˜973 K. The number of hydrogen molecules absorbed by Zr was estimated using the area between the Ar and H₂ curves (shown in FIG. 12A and Table 2) after normalizing flow rates to their corresponding steady state values. Similar hydrogen uptake experiments at varying hydrogen feed pressures (3.28-95.13 kPa, total feed flow rate ˜1.7 cm³ s⁻¹) were performed on a single Zr metal bed and the results are shown in FIG. 12A and Table 2. A bulk stoichiometry of the zirconium hydride (as discussed in Section 3.4.2) resulting from hydrogen uptake was obtained by normalizing the total number of hydrogen atoms absorbed by the number of total Zr metal atoms present in the sample.

As noted in Table 2, the hydrogen breakthrough times decreased with increasing hydrogen pressures and the uptake curves at hydrogen pressures exceeding 10 kPa resembled heavyside functions as shown in FIG. 12A. At ˜973 K, Zr absorbed hydrogen to form ZrH_{1.75} consistently across a large hydrogen pressure range (3.28-95.13 kPa, FIG. 12C and Table 2). These results suggested that the rate of hydrogen uptake on bulk zirconium metal was not kinetically relevant as sigmoidal uptake curves would be expected as hydrogen pressures increased if this were the case. The effect of Zr addition to MoCₓ/H-ZSM-5 catalysts during methane DHA reactions at 973 K was, therefore, limited by the transport of hydrogen from MoCₓ moieties in the zeolite to the Zr metal.

Structural Characterization of Zirconium and Zirconium Hydride.

Following the hydrogen uptake of zirconium metal particles, temperature-programmed-desorption (TPD) in helium flow was performed to investigate the stoichiometry of zirconium hydride formed and the proficiency of zirconium metal for regeneration after hydrogen absorption. Hydrogen was observed to evolve from zirconium hydride particles as the temperature of the sample was increased from ˜973 K to ˜1193 K at ˜0.061 K s⁻¹ and the corresponding time-on-stream evolution for different hydrogen uptake pressures (3.28-95.13 kPa), monitored using an online mass spectrometer, is shown in FIG. 12B. Helium was used as an internal standard to calculate the total amount of hydrogen desorbed from the sample during TPD and the results are presented in Table 2. The results from hydrogen uptake (H_{absorbed}:Zr) and helium TPD (H_{desorbed}:Zr) experiments are collated in FIG. 12C and Table 2. These results demonstrated that (i) zirconium metal absorbs hydrogen at ˜973 K consistently across a large hydrogen pressure range (3.28-95.13 kPa) to form a bulk hydride with stoichiometry ZrH_{1.75} and (ii) all the hydrogen absorbed by Zr metal can be removed by helium TPD at ˜1193 K to regenerate the zirconium metal.

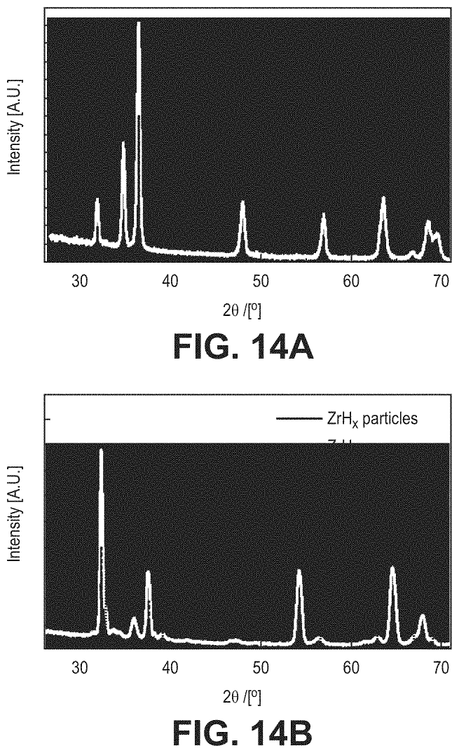

X-ray diffraction patterns of zirconium metal prior to hydrogen uptake and after hydrogen uptake at ˜33.34 kPa hydrogen pressure are shown in FIGS. 14A and 14B, respectively. As shown in FIG. 14A, the reference pattern confirmed that the zirconium material had bulk crystalline characteristics of metallic Zr. Post hydrogen uptake zirconium sample demonstrated mixed hydride phases of stoichiometry ZrH and ZrH_{1.66} which are in close agreement with the hydrogen uptake and temperature-programmed-desorption experiment results noted in Table 2 and FIG. 12C.

Regeneration of CH₄ Dehydroaromatization Catalysts—Regeneration of Physical Mixture of Zr and MoCₓ/H-ZSM-5 for CH₄ DHA.

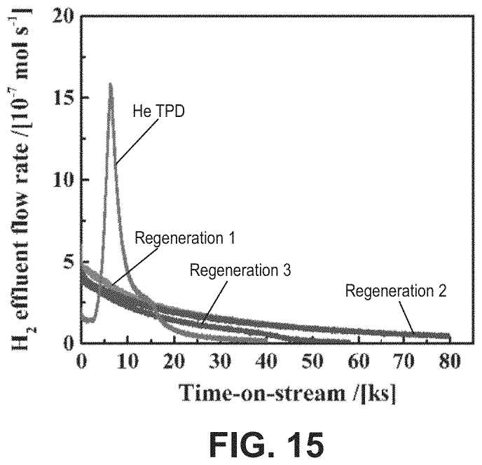

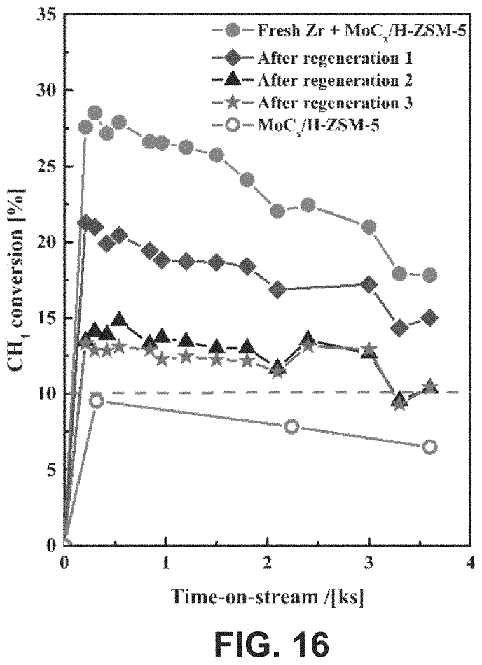

Regeneration of the polyfunctional Zr+MoCₓ/H-ZSM-5 catalyst formulation was investigated by treating the catalyst in helium flow at ˜973 K after performing methane DHA for ˜3.6 ks. During the helium flush at ˜973 K, hydrogen was observed to elute from the catalyst, presumably from the hydrogen absorbed by zirconium during reaction, as monitored using an online mass spectrometer (shown in FIG. 15). Transient effluent flow rate of hydrogen during regeneration of Zr+MoCₓ/H-ZSM-5 catalyst for methane DHA is shown in FIG. 15. Helium was used as an internal standard to quantify the amount of hydrogen eluted during helium flush or post-reaction helium temperature-programmed-desorption (TPD). The amount of hydrogen removed was quantified using helium as an internal standard. Methane dehydroaromatization reactions were again performed post the helium flush and the resulting methane conversion as a function of time-on-stream is shown in FIG. 16. The helium flush resulted in regeneration of the absorptive-hydrogen-function. Consequently, above equilibrium methane conversions (equilibrium conversion ˜10%) were obtained during each reaction-regeneration cycle (˜22-15% maximum conversion) as shown in FIG. 16. The total methane turnover, defined as number of methane molecules converted per Mo atom, at the end of ˜3.6 ks for each regeneration cycle is shown in Table 3 demonstrating that the Zr+MoCₓ/H-ZSM-5 catalyst formulation consistently converted higher amounts of methane (˜10.73-19.8 mol mol_{Mo}⁻¹) as compared to the conventional MoCₓ/H-ZSM-5 catalyst (˜6.14 mol mol_{Mo}⁻¹). Catalyst deactivation in Zr+MoCₓ/H-ZSM-5 during methane reactions was caused not only by loss of hydrogen-absorbing sites due to zirconium hydride formation but was compounded by loss of Brønsted acid sites caused by formation of unsaturated carbonaceous species in the zeolite channels. The regeneration protocol presented in this Example did not regenerate the acid sites which would account for the lack of complete regeneration post helium flush. Hydrogen balance calculations for the reaction-regeneration cycles are reported below and in (Table S1). The total amount of hydrogen removed during successive cycles of helium flow and post-reaction helium TPD (H:Zr˜3.1) as in good agreement with the hydrogen missing in the reactor effluent during the corresponding reaction cycles based on hydrogen balance calculations using equation 5 (H_{missing}:Zr˜2.32). This discrepancy can be attributed to the experimental error in the quantification of hydrogen using an online mass spectrometer at the very low concentrations of hydrogen present in the effluent during catalysis.

H_{missing}=(2×H₂+4×C₂Hₓ+6×C₆H₆+8×C₇H₈+10×C₈H₁₀+8×C₁₀H₈+10×C₁₀⁺)−(4×CH₄ reacted) (5)

Temperature-Programmed-Desorption after CH₄ Dehydroaromatization on Zr+MoCₓ/H-ZSM-5.

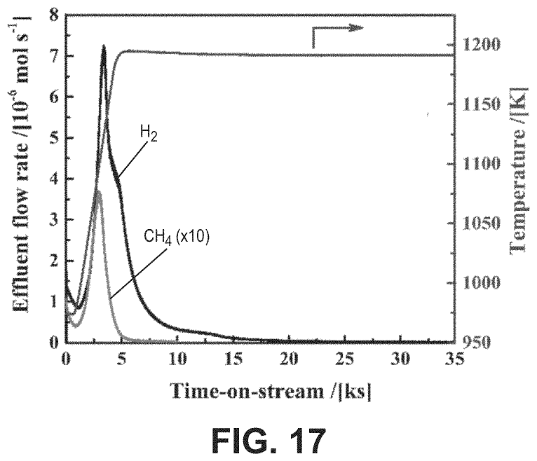

A temperature-programmed-desorption (TPD) in helium flow (˜0.83 cm³ s⁻¹) at ˜1193 K was performed following methane dehydroaromatization reaction at 973 K on an interpellet physical mixture of Zr and MoCₓ/H-ZSM-5 for ˜9 ks (shown in FIGS. 9A-9B) and the corresponding reactor effluent transient monitored using a mass spectrometer is shown in FIG. 17. Hydrogen was observed to elute from the reactor presumably from the zirconium hydride formed by hydrogen absorption during methane reaction. Moreover, hydrogenolysis of the carbonaceous species deposited during methane DHA by the desorbed hydrogen resulted in methane elution during post-reaction TPD. Others have previously noted the removal of carbon deposits by treatment in pure hydrogen at ˜973 K resulting in restoration of initial rates of methane conversion on Mo/H-ZSM-5 formulations. The total amount of hydrogen and methane eluted during TPD was calculated using helium as an internal standard.

The total number of hydrogen atoms absent in the reactor effluent at the end of ˜8.7 ks during methane reaction on Zr+MoCₓ/H-ZSM-5 was estimated using equation 5 and normalized by total Zr atoms present in the catalyst bed. The post-reaction TPD yielded H:Zr˜1.60 which was within ˜10% of the hydrogen missing from hydrogen balance (H_{missing}:Zr˜1.48 as noted in Table 1) suggesting that all the hydrogen absorbed by zirconium particles during methane DHA could be removed by high temperature desorption in line with hydrogen uptake experiments discussed in Section 3.4. Similar calculations for methane removed during TPD resulted in C:Mo˜1.69 which was less than the carbon deposited calculated from carbon balance (C:Mo˜3.46). This difference can be attributed to the insufficient availability of hydrogen to hydrogenolyze all the carbon deposited.

In summary, methane reactions on Mo/H-ZSM-5 catalyst at 973 K resulted in an initial carburization period during which O:Mo˜2.44±0.10 was removed as CO and CO₂ due to the formation of (Mo₂O₅)²⁺ dimers on thermal treatment of MoO₃ and H-ZSM-5 physical mixtures at 973 K as reconciled by Raman spectroscopy. Steady state methane dehydroaromatization on MoCₓ/H-ZSM-5 catalysts at 973 K resulted in a forward rate of benzene synthesis ˜(5.05±0.09)×10⁻⁴ mol s⁻¹ mol_{Mo}⁻¹ with ˜67% benzene and ˜21% naphthalene product selectivity. Maximum methane conversion (˜27%), aromatic synthesis rates, and benzene, naphthalene, toluene, xylene, and C₁₀⁺ product yields (1.4-5.4 times) were enhanced on interpellet physical mixtures of Zr and MoCₓ/H-ZSM-5 as compared to MoCₓ/H-ZSM-5 due to in-situ absorptive-hydrogen removal by zirconium metal thereby lifting the thermodynamic constraints for methane dehydrogenation. A post-reaction helium temperature-programmed-desorption at ˜1193 K resulted in absorbed hydrogen removal (H:Zr˜1.60) from zirconium hydride particles (H:Zr˜1.48 from hydrogen balance) formed during methane reactions. Hydrogen uptake experiments on zirconium metal particles at 973 K demonstrated that Zr forms ZrH_{1.75} across a large hydrogen pressure range (3.28-95.13 kPa) which can be regenerated to Zr metal by high temperature (˜1193 K) helium TPD as also supported by XRD analysis. A post-reaction thermal treatment in helium at 973 K of the polyfunctional Zr+MoCₓ/H-ZSM-5 catalyst formulation led to regeneration of the absorptive-hydrogen removal function resulting in above equilibrium methane conversions (˜22-15%) in successive reaction-regeneration cycles.

TABLE 1
Oxygen removed during carburization, methane converted, product
yields, and hydrogen balance for MoCₓ/H-ZSM-5 and an interpellet
physical mixture of MoCₓ/H-ZSM-5 and Zr metal. MoCₓ/H-ZSM-5
loading ~1.2 g with Mo/A1_{f} ~0.25, Zr metal loading ~2.4 g, total flow
rate ~0.21 cm³ s⁻¹ (90 vol % CH₄/balance Ar), reaction at ~973 K.
Corresponding time-on-stream data shown in FIGS. 5-8.

| | MoCₓ/H-ZSM-5 | Zr + MoCₓ/H-ZSM-5 |
|---|---|---|
| O_{removed}:MO | 2.44 ± 0.1 | 2.51 ± 0.1 |
| (mo1ₒ mo1_{Mo}⁻¹)^{a} | | |
| CH₄ converted (mo1ₒ | 13.68 | 35.85 |
| mo1_{Mo}⁻¹)_{b} | | |
| Product Yield (mo1ₒ | | |
| mo1_{Mo}⁻¹)_{b} | | |
| C₂Hₓ | 0.49 | 0.70 |
| Benzene | 2.07 | 3.39 |
| Toluene | 0.099 | 0.199 |
| Xylenes | 0.014 | 0.200 |
| Naphthalene | 0.284 | 0.602 |
| C₁₀⁺ | 0.028 | 0.150 |
| H₂^{c} | 9.02 | 7.57 |
| H_{missing}:Zr (mo1_{H} mo1_{Zr}⁻¹)^{d} | NA | 1.48 |
| H:Zr (helium TPD) (mo1_{H} | NA | 1.60 |
| mo1_{zr}⁻¹)^{e} | | |

^{a}number of oxygen atoms removed per Mo atom calculated considering oxygen-containing products (CO and CO₂) eluted from the reactor during the initial 15.5 ks of methane reaction on Mo/H-ZSM-5 as shown in FIG. 2A.
^{b}cumulative methane converted or product yields calculated after 8.7 ks time-on-stream as shown in FIG. 8.
^{c}hydrogen (H₂) yield per Mo atom calculated from time-on-stream data shown in FIG. 7(f)
^{d}number of hydrogen atoms missing in the reactor effluent per Zᵣ atom at the end of 8.7 ks time-on-stream, calculated as: H_{missing} = (2 × H₂ + 4 × C₂Hₓ + 6 × C₆H₆ + 8 × C₇H₈ + 10 × C₈H₁₀ + 8 × C₁₀H₈ + 10 × C⁺₁₀) − (4 × CH4 reacted) where, effluent flow rates are used for all products and CH₄ converted is calculated as (CH_{4 in} − CH_{4 out}) during reaction
^{e}number of hydrogen atoms per Zr atom eluted during temperature-programmed-desorption (TPD) in helium flow (~0.83 cm³ s⁻¹) at ~1193 K, following the methane reaction on interpellet physical mixture of MoCₓ/H-ZSM-5 and Zr metal, as shown in FIG. 12A-12C.

TABLE 2
Experimental conditions and measured hydrogen uptake during hydrogen
uptake experiments for Zr metal shown in FIG. 9A-9B. Zr metal loading
~2.4273 g (~0.0267 moles), Zr particle diameter ~3 × 10⁻⁴ m.

| H₂ flow rate/ | H₂ uptake | Breakthrough | H₂ absorbed/ | H₂ desorbed/ | H absorbed: | H desorbed: |
|---|---|---|---|---|---|---|
| (10⁻⁶ mol s⁻¹)^{a} | pressure (kPa) | time (ks)^{b} | (¹⁰⁻² moles)^{c} | (¹⁰⁻² moles)^{d} | Zr^{e} | Zr^{f} |
| 2.80 | 3.28 | 6.4 | 2.27 | 2.34 | 1.71 | 1.75 |
| 8.14 | 11.64 | 1.9 | 2.32 | 2.38 | 1.74 | 1.79 |
| 33.34 | 51.74 | 0.6 | 2.42 | 2.41 | 1.81 | 1.81 |
| 61.92 | 95.13 | 0.3 | 2.29 | 2.28 | 1.72 | 1.71 |

^{a}hydrogen flow rate during hydrogen uptake experiment for Zr metal shown in FIG. 9A, total feed flow rate ~1.7 cm³ s⁻¹ (balance Ar), temperature ~973 K.
^{b}time delay for hydrogen signal in mass spectrometer with respect to Ar signal (internal standard and tracer) as shown in FIG. 9A.
^{c}number of moles of hydrogen absorbed by Zr metal, calculated using the breakthrough time and the transient during hydrogen uptake experiment shown in FIG. 9A.
^{d}number of moles of hydrogen eluted from the reactor during temperature-programmed-desorption (TPD) in helium flow (~0.83 cm³ s⁻¹) at ~1193 K as shown in FIG. 9B, following hydrogen uptake shown in FIG. 9A).
^{e}number of hydrogen atoms per Zr atom absorbed during hydrogen uptake (FIG. 9A).
^{f}number of hydrogen atoms eluted per Zr atom during temperature-programmed-desorption (TPD) in helium flow shown in FIG. 9B.

TABLE 3
Methane converted for MoCₓ/H-ZSM-5 and an interpellet physical mixture of
MoCₓ/H-ZSM-5 and Zr metal before and after regeneration in He flow at ~973 K.
MoCₓ/H-ZSM-5 loading ~1.2 g with Mo/A1_{f} ~0.25, Zr metal loading ~2.4 g, total flow
rate ~0.21 cm³ s⁻¹ (90 vol % CH₄/balance Ar), reaction at ~973 K. Corresponding time-
on-stream data shown in FIG. 11A-G.

| | | | Zr + MoCₓ/H-ZSM-5 | | |
|---|---|---|---|---|---|
| | MoCₓ/H- | | After | After | After |
| | ZSM-5 | Fresh | Regeneration 1^{b} | Regeneration 2^{b} | Regeneration 3^{b} |
| CH4 | 6.14 | 19.8 | 13.75 | 11.2 | 10.73 |
| converted | | | | | |
| (mo1 mo1ₘₒ⁻¹)^{a} | | | | | |

^{a}cumulative methane converted calculated after 3.6 ks time-on-stream as shown in FIG. 11A-11G.
^{b}regenerations 1, 2, and 3 of Zr + MoCₓ/H-ZSM-5 were performed by flushing the catalyst in helium flow (~0.83 cm³ s⁻¹) at 973 K for 61.2 ks, 84.6 ks, and 34.2 ks, respectively.

TABLE S1
Methane converted, product yields, hydrogen balance for MoCₓ/H-ZSM-5 and
interpellet physical mixture of MoCₓ/H-ZSM-5 and Zr metal before and after regeneration
in He flow at ~973 K. MoCₓ/H-ZSM-5 loading ~1.2 g with Mo/A1_{f} ~0.25, Zr metal
loading ~2.4 g, total flow rate ~0.21 cm³ s⁻¹ (90 vol % CH₄/balance Ar), reaction at ~973 K.
Corresponding time-on-stream methane conversion data shown in FIG. 11A-11G.

| | | | Zr + MoCₓ/H- | | |
|---|---|---|---|---|---|
| | | | ZSM-5 | | |
| | | | After | After | After |
| | MoCₓ/H-ZSM-5 | Fresh | Regeneration 1^{b} | Regeneration 2^{b} | Regeneration 3^{b} |
| CH4 converted | 6.14 | 19.8 | 13.75 | 11.2 | 10.73 |
| (mo1 mo1ₘₒ⁻¹)^{a} | | | | | |
| Product Yield | | | | | |
| (mo1 mo1ₘₒ⁻¹)c | | | | | |
| C₂Hₓ | 0.19 | 0.214 | 0.213 | 0.216 | 0.210 |
| Benzene | 0.864 | 1.545 | 1.266 | 0.887 | 0.640 |
| Toluene | 0.04 | 0.103 | 0.105 | 0.087 | 0.071 |
| Xylenes | 0.005 | 0.106 | 0.104 | 0.067 | 0.045 |
| Naphthalene | 0.126 | 0.356 | 0.171 | 0.091 | 0.053 |
| C₁₀⁺ | 0.013 | 0.085 | 0.051 | 0.017 | 0.008 |
| H₂^{d} | 9.02 | 3.5 | 3.17 | 2.18 | 1.64 |
| H_{missing}:Zr | NA | 0.83 | 0.53 | 0.47 | 0.49 |
| (mo1_{H} mo1_{Zr}⁻¹)^{e} | | | | | |
| H:Zr (helium | NA | 0.84^{f} | 0.88^{f} | 0.71^{f} | 0.67^{g} |
| flush/TPD) | | | | | |
| (mo1_{H} mo1_{Zr}⁻¹) | | | | | |

^{a}cumulative methane converted calculated after 3.6 ks time-on-stream as shown in FIG. 11A-11G.
^{b}regeneration 1, 2, and 3 of Zr + MoCₓ/H-ZSM-5 were performed by flushing the catalyst in helium flow (~0.83 cm³ s⁻¹) at 973 K for 61.2 ks, 84.6 ks, and 34.2 ks, respectively.
^{c}cumulative product yields calculated after 3.6 ks time-on-stream.
^{d}hydrogen (H₂) yield per Mo atom calculated from time-on-stream mass spectrometer data.
^{e}number of hydrogen atoms missing in the reactor effluent per Zr atom at the end of 3.6 ks time-on-stream, calculated as: H_{missing} = (2 × H₂ + 4 × C₂Hₓ + 6 × C₆H₆ + 8 × C₇Hₛ + 10 × CₛH₁₀ + 8 × C₁₀Hₛ + 10 × C⁺₁₀) − (4 × CH₄ reacted) where, effluent flow rates are used for all products and CH₄ converted is calculated as (CH₄ᵢₙ − CH₄ₒᵤₜ) during reaction
^{f}number of hydrogen atoms per Zr atom eluted during helium flush in helium flow (~0.83 cm³ s⁻¹) at ~973 K, following the methane reaction on interpellet physical mixture of MoCₓ/H-ZSM-5 and Zr metal.
^{g}number of hydrogen atoms per Zr atom eluted during temperature-programmed-desorption (TPD) in helium flow (~0.83 cm3 s−1) at ~1193 K, following the methane reaction on interpellet physical mixture of MoCₓ/H-ZSM-5 and Zr metal.

### Example 2

The following Example relates to controlling kinetic and diffusive length-scales during absorptive hydrogen removal in methane dehydroaromatization on MoCₓ/H-ZSM-5 catalysts.

Addition of Zr metal absorbent to MoCₓ/H-ZSM-5 in the form of staged-bed, stratified-bed, and interpellet physical mixtures effectively scavenged H₂ from catalyst proximity, enhancing maximum single-pass benzene and naphthalene yield during methane dehydroaromatization (DHA) reactions to 14-16% compared to 8% in formulations without zirconium. The coupling of spatially-distinct catalytic and absorptive functions was achieved by dispersive/diffusive transport which conveyed H₂ to staged Zr both co- and counter-current to bulk advection, thereby suppressing axial H₂ partial pressure profiles along the catalyst bed and enhancing net aromatization rates. The significance of dispersive hydrogen transport during methane DHA was shown for the first time by measurement of Péclet number, Pe=1.32, in H₂ tracer studies with step-change or impulse input to inert catalyst proxies. Kinetic limits to methane pyrolysis were quantified by Damköhler number, Da, for synthesis of benzene, Da_{B}=0.15, and naphthalene, Da_{N}=0.03, determined from kinetic studies which rigorously accounted for reversibility of DHA reactions. Detailed reaction-transport models synthesized interplay of kinetic, diffusive, and convective length-scales captured by Péclet and Damköhler numbers to predict influence of catalyst-absorbent proximity and process flow-conditions on aromatization rates. Systematic control of catalyst bed-length, L, or linear flow velocity, u, predictably altered Pe and Da to effect improvements in methane conversion with and without Zr metal, corroborating results from simulation of the reaction-transport model.

### INTRODUCTION

Dehydroaromatization (DHA) of methane to benzene (6CH₄↔C₆H₆+9H₂) is highly endothermic and requires reaction temperatures≥950 K to achieve about 10% equilibrium conversion at ambient pressure. Carbidic forms of Mo encapsulated in medium-pore MFI zeolites catalyze methane DHA with high benzene selectivity (about 70%) at conversions near the thermodynamic limit. Oxidic Mo precursors, deposited either by aqueous impregnation or stoichiometric exchange with zeolitic H⁺ pairs, reduce and carburize upon exposure to CH₄ at high temperatures (≥950 K) to form molecular-size MoCₓ clusters (0.6-1.5 nm) which activate C—H bonds in methane to initiate C—C coupling reactions that lead to aromatics. Oxygen removed (2.44±0.1 O:Mo) in the form of H₂O, CO, and CO₂ during reduction/carburization procedures corroborated formation of putative (Mo₂O₅)²⁺ dimers which are anchored to proximal Al site pairs on MoOₓ/H-ZSM-5 materials prepared by solid-state stoichiometric exchange. Carbon deposition of about 9 C:Mo concurrent with oxygen evolution during catalyst reduction/carburization is the predominant source of coke formation on materials with about 3 wt % Mo.

H₂ formed during dehydrogenation and cyclization events on Mo/H-ZSM-5 inhibits net synthesis rates during methane pyrolysis; in-situ removal of hydrogen from catalyst proximity is virtuous in overcoming thermodynamic barriers of methane DHA set by reaction endothermicity. It has been demonstrated that permselective membrane walls integrated into CH₄ DHA flow reactors effectively scavenged liberated hydrogen, modestly enhancing methane conversion from about 10% to about 12%. However, improvement in methane pyrolysis rates was principally owed to increased formation of entrained C₁₂₊ polynuclear aromatics as product distribution shifts to higher molecular weight unsaturated hydrocarbons which benefit most from hydrogen removal. The accumulation of large polycyclic aromatics congests zeolite pores and leads to a more rapid decrease in instantaneous conversion—first-order deactivation rate constants increase from 0.044 h⁻¹ to 0.059 h⁻¹ upon integration of permselective membrane walls.

Example 1 demonstrates that in-situ hydrogen removal can be achieved by introduction of a selective hydrogen absorbent, such as zirconium metal. Addition of Zr metal particles to MoCₓ/ZSM-5 in interpellet mixtures resulted in a 2.7× increase in methane converted and concurrent 1.4, 2.1, 2.6, and 5.6-fold increase in effluent benzene, naphthalene, toluene, and C₁₀₊ yields, respectively, in reference to a conventional MoCₓ/ZSM-5 catalyst at equivalent time-on-stream (8.7 ks). The maximum methane conversion on interpellet mixtures exceeded 27%—in significant excess of about 10% equilibrium-prescribed conversion at these reaction conditions—demonstrating that thermodynamic limitations encountered in methane dehydroaromatization on MoCₓ/ZSM-5 are circumvented by addition of Zr as a hydrogen absorbent. Characterization of Zr metal via hydrogen uptake experiments and X-ray diffraction evinced formation of ZrH_{1.75} on hydrogen exposure (3.28-95.13 kPa) at 973 K.

In this Example 2, the critical role of catalyst-absorbent proximity and reactor hydrodynamics in determining efficacy of polyfunctional mixtures to achieve supra-equilibrium aromatic yields in methane DHA reactions is disclosed. H₂ tracer studies with impulse and step-change inputs to flow vessels charged with inert catalyst proxies revealed fluid flow was highly dispersed under reaction flow conditions. Measurement of axial dispersion coefficients, kinetic orders, and forward rate constants formed the foundation of detailed reaction-transport models used to simulate DHA reactions with and without Zr addition. Péclet number, Pe, and Damköhler number, Da, arose naturally from non-dimensionalization of governing transport equations and provided compendious metrics to evaluate significance of kinetic, diffusive, and convective length-scales. Interplay of kinetic and transport length-scales was studied by systematic change of catalyst-absorbent intimacy and flow conditions in both experimental investigations and model simulations. Reaction-transport models corroborate observed proximity effects and provided a well-founded mathematical framework to rationalize influence of reactor hydrodynamics captured by Péclet and Damköhler numbers.

### Materials and Methods

2.1. Catalyst Synthesis and Preparation.

Mo/H-ZSM-5, MoCₓ/H-ZSM-5, and polyfunctional configurations of MoCₓ/H-ZSM-5+Zr were prepared by methods detailed in Example 1 and summarized herein. Briefly, NH₄-ZSM-5 (Zeolyst International, Si/Al=11.5, CBV 2314) was converted to H-ZSM-5 by treating in dry air (about 1.67 cm³ s⁻¹) to thermally decompose NH₄⁺ to H⁺ and NH₃ (g) by increasing the temperature from ambient temperature to 773 K at 0.0165 K s⁻¹ and holding at 773 K for 36 h. Intimate mixtures containing a nominal ˜3 wt % Mo loading (Mo/Al≈0.25) were prepared by grinding together MoO₃ (Sigma-Aldrich, 99.9%) and H-ZSM-5 powders in an agate mortar and pestle for 0.25 h. The mixture was heated from ambient temperature to 623 K at 0.0167 K s⁻¹ and held at this temperature for 15 h in dry air (ca. 0.67 cm³ s⁻¹) resulting in water removal and dispersion of MoO₃ on the external surface of the zeolite. Finally, the mixture was heated to 973 K at 0.167 K s⁻¹ and held at this temperature for 10 h to facilitate molybdenum oxide migration into the zeolite pores. The catalyst prepared via the above-mentioned protocol is referred to as Mo/H-ZSM-5 in this Example. The Mo/H-ZSM-5 powder was pressed to form pellets that were then crushed and sieved to obtain particle sizes between 180 and 425 mm (mesh 40-80) for use in catalytic reactions.

The samples designated as MoCₓ/H-ZSM-5 in this Example were prepared by exposing the Mo/H-ZSM-5 formulation to a CH₄/Ar mixture (90 vol % CH₄ and 10 vol % Ar, total feed flow rate ca.0.27 cm³ s⁻¹) for 15.5 ks (for catalyst loading ˜1.2 g) leading to complete carburization of the molybdenum oxide as described in Example 1. Subsequently, the feed was switched to helium (ca. 0.83 cm³ s⁻¹). The sample was flushed in helium flow (ca. 0.83 cm³ s⁻¹) at 973 K for 0.9 ks and then cooled to ambient temperature in the same helium flow.

Zirconium (Zr) metal was obtained from American Elements (PN # ZR-M-0251 M-GR.1T2MM, 99.5+% purity, metal basis) as granule-shaped particles (1-2 mm granule size) which were crushed and sieved to obtain particle sizes between 180 and 425 mm (mesh 40-80) for use in the catalytic reactions.

All polyfunctional configurations of MoCₓ/H-ZSM-5 and Zr were constituted in an inert environment using a glove bag (Sigma Aldrich, Z530212, AtmosBag, two-hand, non-sterile, size M, Zipper-lock closure type) to eliminate oxidation of MoCₓ species under ambient conditions. This procedure gave five fixed-bed configurations: (i) MoCₓ/H-ZSM-5 only, (ii) Zr packed upstream of the MoCₓ/H-ZSM-5 catalyst bed, (iii) Zr packed downstream of the MoCₓ/H-ZSM-5 catalyst bed, (iv) Zr packed both upstream and downstream of the MoCₓ/H-ZSM-5 catalyst bed (referred to as sandwich configuration), and (v) an interpellet mixture of MoCₓ/H-ZSM-5 and Zr metal. Reconfigured catalyst absorbent mixtures were subsequently loaded in the same quartz reactor under an inert atmosphere and transferred to the furnace without exposure to ambient conditions. The reactor was heated to the reaction temperature, 973 K, in helium flow (ca. 0.33 cm³−s⁻¹) at 0.18 K s⁻¹ before performing methane dehydroaromatization reactions.

2.2. Methane Dehydroaromatization Catalytic Reactions.

Methane dehydroaromatization reactions were performed in a fixed bed tubular quartz reactor (I.D. 10.5 mm). The catalyst sample was heated in helium flow (ca. 0.33 cm³ s⁻¹, UHP, Minneapolis Oxygen) from ambient temperature to the reaction temperature, 973 K, at 0.18 K s⁻¹ in a resistively heated furnace (National Element FA120). Axial temperature gradients in the reactor were minimized by using an annular inconel cylinder to ensure conduction of heat between walls of the furnace and the tubular reactor. The temperature of the catalyst sample was monitored by two thermocouples stationed diametrically on the exterior wall of the quartz reactor. A feed gas mixture of CH₄/Ar (90 vol % CH₄ and 10 vol % Ar, total feed flow rate ca. 0.27 cm³ s⁻¹, UHP, Matheson Tri-Gas) was introduced to the reactor to perform methane reactions at 973 K and atmospheric pressure, with Ar serving as an internal standard. All flow lines were heated to temperatures in excess of 450 K via resistive heating to prevent condensation of effluents. The composition of the reactor effluent was analyzed using a mass spectrometer (MKS Cirrus 200 Quadrupole MS system) and a gas chromatograph (Agilent 7890) equipped with a methyl-siloxane capillary column (HP-1, 50 m×320 mm×0.52 mm) connected to a flame ionization detector (FID) for detection of hydrocarbons and a GS-GasPro column (60 m×0.320 mm) connected to a thermal conductivity detector (TCD) for detection of permanent gases (H₂, Ar, and CH₄). Transient product evolution throughout the course of the reaction was measured with an online mass spectrometer (MS) (MKS Cirrus 200 Quadrupole MS system) connected to the outlet of the GC. The number of O atoms removed during carburization of molybdenum oxide was determined by the cumulative amount of H₂O, CO, and CO₂ eluted as calculated from the transient MS signal with Ar as an internal standard.

Evaluation of methane DHA kinetic orders and rate constants was performed on MoCₓ/H-ZSM-5 in the regime when benzene net rate was nearly invariant with time-on-stream (15-25 ks time-on-stream for 1.2 g catalyst loading). H₂ (0-0.23 kPa at 97.6 kPa CH₄ and balance argon, total flow rate ca. 0.27 cm³ s⁻¹ and catalyst loading=1.2 g) and methane (2.7-97.6 kPa, balance argon, total flow rate ca. 0.27 cm³ s⁻¹ and catalyst loading=1.2 g) partial pressures were varied at constant contact time by altering H₂, inert (Ar), and methane flow rates.

2.3. Measurement of Axial H₂ Diffusion Coefficient in Packed-Bed Flow Reactors.

Inert H₂ tracer experiments were performed on beds of 180-425 μm quartz sand aggregates packed to 2.7 cm, an identical length as 1.2 g of 180-425 μm MoCₓ/H-ZSM-5 aggregates in a 1.05 cm ID tubular quartz reactor. Quartz sand was heated to 973 K in inert (He or Ar) flow (ca. 1 cm³ s⁻¹). For pulse tracer experiments, a sample of H₂ (ca. 1 cm³) was introduced to a stable reactor feed flow of CH₄ using an electronic six-way valve (ED66UWE, VICI Valco 6-port valve). In step-change experiments, reactor feed flow was switched from Ar to an Ar and H₂ gas mixture (5 vol % H₂/balance Ar). Reactor effluent was monitored using an online mass spectrometer (MS) (MKS Cirrus 200 Quadrupole MS system).

### Results and Discussion

Rates of non-oxidative methane conversion to benzene, naphthalene, methylarenes, and C2 hydrocarbons were measured over 1.2 g of MoCₓ/H-ZSM-5 catalysts (Mo/Al_{f}=0.25) with and without 2.4 g of Zr metal at a temperature of 973 K and 13.0 cm³ s⁻¹ flow of 90%/10% CH₄/Ar in five reactor configurations listed in Section 2.1.

3.1. Methane DHA on MoCₓ/H-ZSM-5 in Absence and Presence of Zr Metal.

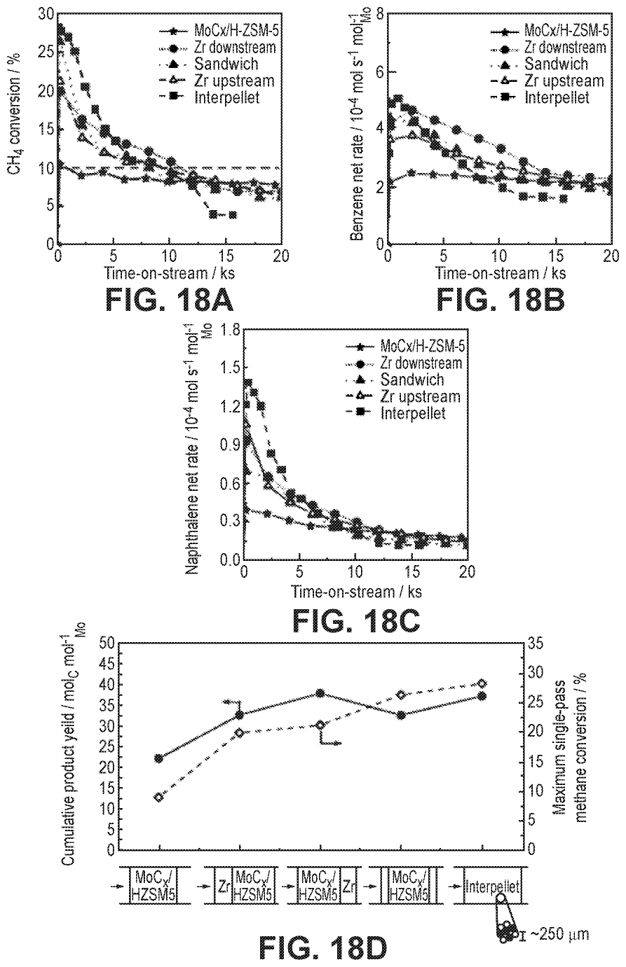

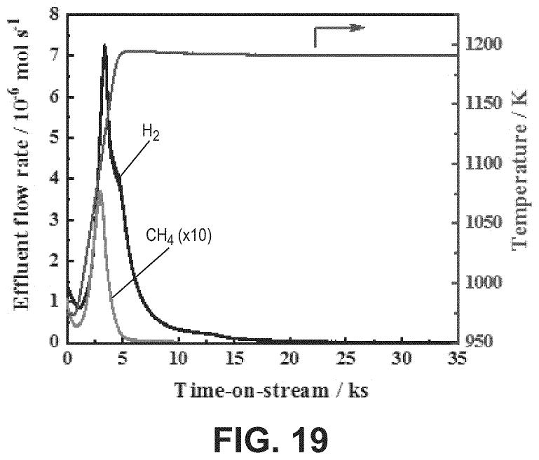

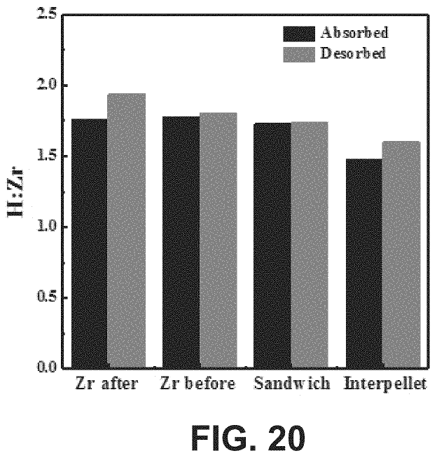

FIGS. 18A-18D shows methane conversion, benzene net rates, and naphthalene net rates as a function of time-on-stream for the five MoCₓ/H-ZSM-5+Zr fixed-bed configurations at 973 K and atmospheric pressure.Zr metal beds introduced downstream, upstream, and both downstream and upstream of the MoCx/H-ZSM-5 catalyst continuously abstract H₂, resulting in circumvention of thermodynamic limits and maximum methane conversion to ˜21%, ˜20%, and ˜26%, respectively, as compared to ˜9% for MoCx/H-ZSM-5 only (FIG. 18A). Improvement to total methane conversion is reflected by accelerated benzene and naphthalene net rates as shown in FIG. 18B-18C. MoCx/H-ZSM-5+Zr catalyst systems in downstream, upstream, sandwich, and interpellet configurations gave maximum benzene net rates of 4.7×10⁻⁴ mol s⁻¹ mol_{Mo}⁻¹, 3.8×10⁻⁴ mol s⁻¹ mol_{Mo}⁻¹, 4.4×10⁻⁴ mol s⁻¹ mol_{Mo}⁻¹, and 5.1×10⁻⁴ mol s⁻¹ mol_{Mo}⁻¹, respectively, in contrast to 2.5×10⁻⁴ mol s⁻¹ mol_{Mo}⁻¹ for MoCₓ/H-ZSM-5 as shown in FIG. 18B. Naphthalene net rates were maximized at 0.93×10⁻⁴ mol s⁻¹ mol_{Mo}⁻¹, 1.06×10⁻⁴ mol s⁻¹ mol_{Mo}⁻¹, 0.70×10⁻⁴ mol s⁻¹ mol_{Mo}⁻¹, and 1.39×10⁻⁴ mol s⁻¹ mol_{Mo}⁻¹ for downstream, upstream, sandwich, and interpellet configurations, respectively, as opposed to 0.40×10⁻⁴ mol s⁻¹ mol_{Mo}⁻¹ for MoCx/H-ZSM-5 (FIG. 18C). Methane conversion and aromatic product rates decreased with time-on-stream (FIGS. 18A-18C) due to both loss of catalytic active sites on MoCx/H-ZSM-5 and saturation of hydrogen absorption sites on/in Zr metal. Section S1 details post-reaction (see FIG. 19) and in-situ (see FIG. 20) quantification of hydrogen absorbed by zirconium metal in all fixed-bed formulations.

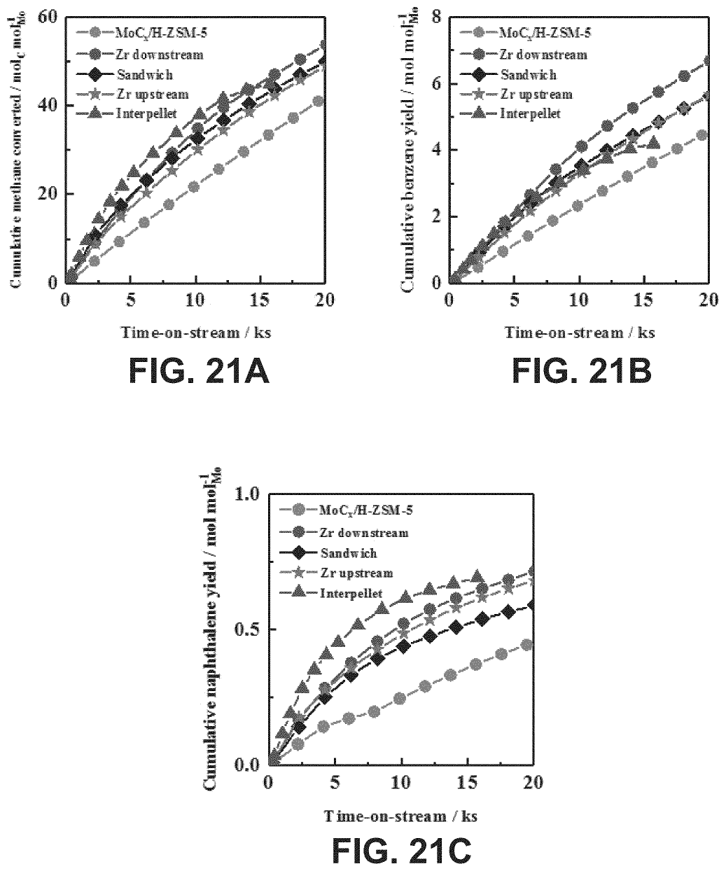

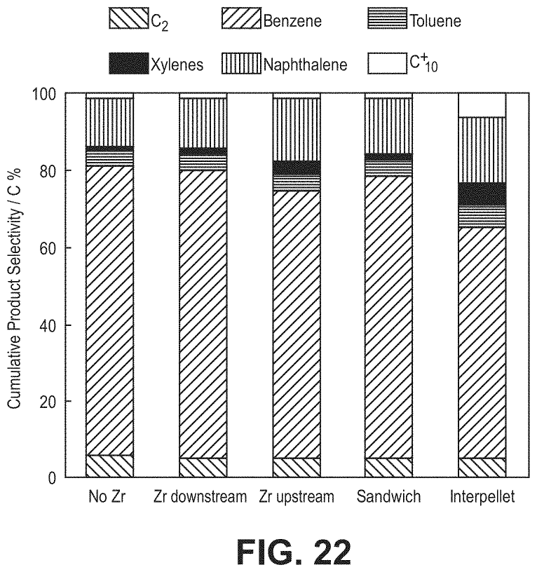

Cumulative product yield at time-on-stream, t, is defined as the total number of moles of product formed per total moles of Mo in the catalyst bed at the end of time, t, and is obtained from the area under the curve of the net product formation rate vs. time-onstream plots shown in FIGS. 18B-18C. Cumulative product yields for methane DHA at 973 K, considering C₂Hₓ (ethane, ethylene, and acetylene), benzene, toluene, xylenes, naphthalene, and C₁₀⁺ aromatics, were calculated for the five fixed-bed configurations at 10.2 ks time-on-stream; their carbon sum (denoted as cumulative product yield in mol_{C} mol_{Mo}⁻¹) is shown in FIG. 18D. Time-on-stream variation of benzene and naphthalene cumulative yields for different catalyst-bed configurations is shown in FIGS. 21A-21C. MoCx/H-ZSM-5+Zr staged-bed configurations resulted in a 1.5, 1.7, 1.5, and 1.7-fold increase in cumulative product yields for Zr bed packed upstream of MoCx/H-ZSM-5, downstream of MoCx/H-ZSM-5, sandwich configuration, and an interpellet mixture, respectively, as compared to MoCx/H-ZSM-5 (FIG. 18D). Catalytic reaction pathways over MoCx/H-ZSM-5 are unperturbed by addition of Zr metal, demonstrated by invariant cumulative aromatic product selectivity of ˜70% benzene and ˜20% naphthalene in all Zr+MoCx/H-ZSM-5 configurations, shown in FIG. 22.

These results demonstrated that irrespective of spatial intimacy between catalyst and Zr metal, the polyfunctional MoCₓ/H-ZSM-5 and Zr absorbent formulation maintains efficacy to enhance methane conversion without detriment to aromatic selectivity. Ability of staged/stratified zirconium to continuously abstract hydrogen and effect improvement to net rates demonstrates gas phase H₂ is conveyed dispersively to Zr absorbent both upstream and downstream of catalyst under reaction conditions. Significance of dispersive transport considerations and influence of catalyst absorbent proximity can be rigorously described by differential mole balance on catalyst and absorbent beds. The resultant reaction-transport model, presented in Section 3.3, requires determination of reaction rate equations and measurement of relevant kinetic and transport parameters, as discussed next in Section 3.2.

3.2. Evaluation of Methane DHA Kinetic and Transport Parameters on MoCx/H-ZSM-5—3.2.1. Kinetics of Methane DHA on MoCx/H-ZSM-5.

Kinetic measurements for methane DHA were performed on pre-carburized MoCx/H-ZSM-5 formulations after exposing Mo/HZSM-5 (1.2 g catalyst loading) to CH₄/Ar mixtures (90 vol % CH₄/balance Ar) for 15.5 ks. The forward rate of benzene and naphthalene formation for the stoichiometric reactions of CH₄ to H₂ and C₆H₆(Eq. (1)) and CH₄ to H₂ and C₁₀H₈ (Eq. (2)) on MoCx/HZSM-5 at 973 K were obtained using a methodology reported previously.

CH₄↔⅙C₆H₆+ 3/2H₂ (1)

CH₄↔ 1/10C₁₀H₈+ 8/5H₂ (2)

Briefly, the net rate of benzene and naphthalene formation and approach to equilibrium of benzene (B) and naphthalene (N) was utilized,

$$\begin{matrix}{η}_{B} = \frac{{P}_{B}^{\frac{1}{6}} {P}_{{H}_{2}}^{\frac{3}{2}}}{{P}_{M} {K}_{B} {P}^{\frac{2}{3}}} & ( 3 ) \\ {η}_{N} = \frac{{P}_{N}^{\frac{1}{10}} {P}_{{H}_{2}}^{\frac{8}{5}}}{{P}_{M} {K}_{N} {P}^{\frac{2}{10}}} , & ( 4 )\end{matrix}$$

calculated from gas-phase total pressure, P, partial pressures, Pⱼ, and equilibrium constants K_{B} and K_{N}, to quantify forward rates of benzene formation, \vec{r_{B}}=(5.05±0.09)×10⁻⁴ mol s⁻¹ mol_{Mo}⁻¹, and naphthalene formation, \vec{r_{N}}=(0.99±0.01)×10⁻⁴ mol s⁻¹ mol_{Mo}⁻¹, at 973 K, 90 vol % CH₄/balance Ar, and 1 atm total pressure. The measured forward rate of benzene formation was similar to rates reported by others (4.7±0.8×10⁻⁴ mol s⁻¹ mol_{Mo}⁻¹) and (5.03×10⁻⁴ mol s⁻¹ mol_{Mo}⁻¹) for methane DHA on Mo/H-ZSM-5 at 950 K.

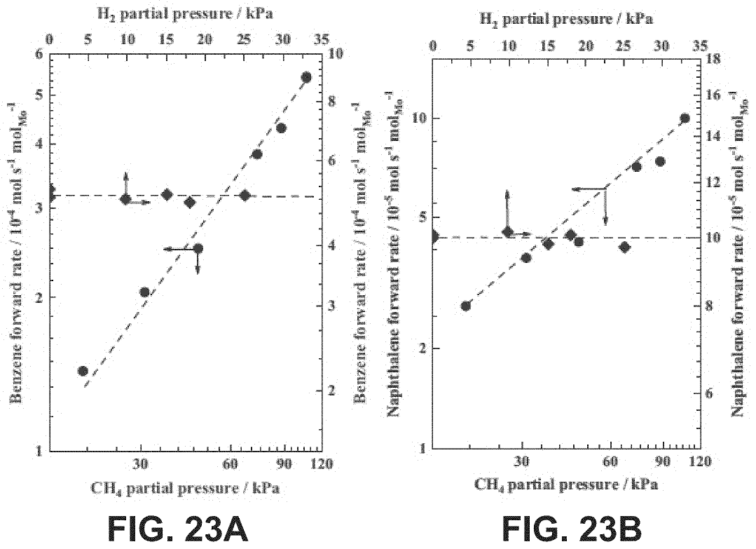

FIGS. 23A-23B shows H₂ and methane partial pressure dependence of benzene and naphthalene forward rates over MoCₓ/H-ZSM-5 catalysts. Forward rates of benzene formation ((5.2±0.05)×10⁻⁴ mol s⁻¹ mol_{Mo}⁻¹) and naphthalene formation ((0.99±0.02)×10⁻⁴ mol s⁻¹ mol_{Mo}⁻¹) were independent of hydrogen co-feed partial pressure (0-23 kPa) demonstrating that hydrogen does not have any kinetic effect on the rate-limiting step of CH₄ DHA. These results are in line with the invariant forward rate of benzene formation ((3.8±0.5)×10⁻⁴ mol s⁻¹−mol_{Mo}⁻¹) at different Mo/H-ZSM-5 catalyst loadings (0.1-1.0 g) and H₂:CH₄ co-feeds (0.03-0.11 M ratio) for methane DHA at 950 K reported previously. Others demonstrated that the net rate of benzene formation systematically decreased with increasing H₂ co-feed concentration (H₂:CH₄=0.09-0.27 M ratio) and that benzene net rates recovered to their pre-H₂ co-feed values upon removal of H₂ from the reactor influent evincing that hydrogen does not cause any irreversible structural or chemical modification to the MoCx moieties present in the catalyst during methane DHA at 950 K.

Benzene similarly has no kinetic effect on the stoichiometric reaction of CH₄ to H₂ and C₆H₆(Eq. (1)) on Mo/H-ZSM-5 at 950 K as evident from an invariant benzene forward rate ((3.8±0.5)×10⁻⁴ mol s⁻¹ mol_{Mo}⁻¹) at varying outlet benzene partial pressure (0.1-0.6 kPa) achieved via different catalyst loadings (0.05-1.0 g Mo/H-ZSM-5). It was presumed that naphthalene also had no kinetic effect on aromatization rates. Methane kinetic orders for benzene and naphthalene formation are ˜0.72 and ˜0.74 (FIGS. 23A-23B), respectively, for methane partial pressure 2.7-97.6 kPa on MoCx/H-ZSM-5 at 973 K implying forward rates of benzene and naphthalene can be expressed by the rate equations shown in Eqs. (5) and (6). These rate equations were utilized in conjunction with the Péclet number measurements (discussed in Section 3.2.2 below) to develop a reaction-transport model for methane DHA on MoCx/H-ZSM-5+Zr catalyst formulations.

\vec{r_{B}}+k_{B}P_{CH}_{₄}^{0.72} (5)

\vec{r_{N}}+k_{N}P_{CH}_{₄}^{0.74} (6)

3.2.2. Measurement and Calculation of Péclet Number, Pe.

The observed effects of catalyst-absorbent proximity on aromatic synthesis rates during methane DHA reactions, particularly those noted upon introduction of Zr upstream and/or downstream of the catalyst bed, highlight the critical role of dispersive H₂ transport in polyfunctional configurations. Efficacy of staged Zr beds to enhance aromatic yields to a similar degree as in interpellet mixtures demonstrates gas-phase H₂ generated over Mo/H-ZSM-5 catalysts is capable of bed-scale transport both co- and counter-current to bulk advection. Relative contributions of diffusive/dispersive and convective transport in tubular flow reactors can be assessed based on a Péclet number

$$Pe = uL / {D}_{eff} = ( \frac{{u}_{s}}{ɛ} ) L / {D}_{eff} , \qquad ( 7 )$$

which is the non-dimensional ratio of characteristic rates of diffusive and convective gas-phase transport in tubular flow reactors. Pe provides a useful metric to evaluate significance of axial dispersion considerations in terms of catalyst bed-length, L, effective diffusion coefficient, D_{eff}, and total linear flow velocity, u, or superficial linear flow velocity, uₛ, and catalyst bed void fraction, E. Ideal plug flow reactors (PFRs) operate in the limit of Pe→∞ wherein unidirectional bulk advection is the sole mechanism of mass transport, thereby rendering neighboring axial fluid-elements incapable of exchanging matter. Axial dispersion becomes relevant for Pe which permits bed-scale molecular transport in response to concentration gradients arising from chemical reaction and/or absorption rate processes either (pseudo)-homogenously or at reactor bounds.

Introduction of catalyst or absorbent packed-beds in tubular flow reactors obstruct molecular movement, thereby shortening mean-free path and dampening diffusional motion. Consequently, effective bulk gas-phase diffusion coefficients, D_{eff}, decrease compared to the molecular limit, D, which defines proportionality between diffusive flux and species concentration gradients in an unobstructed fluid medium. Substitution of D for D_{eff}t into Eq. (7) provides an absolute lower-bound on Pe. D_{eff}, and Pe were evaluated by theoretical and experimental means to corroborate observations of bed-scale hydrogen diffusion during methane dehydroaromatization reactions in polyfunctional catalyst+absorbent formulations.

Levenspiel details systematic procedures for approximation of Péclet number for conditions which deviate from the ideal plug flow reactor. Calculation of Bodenstein number

Bo=ud/D, (8)

and catalyst-bed aspect-ratio,

A=L/d, (9)

where d is reactor diameter, give Bo=0.12 and A=2.57 and suggest reactor configurations discussed in this investigation are neither ideally-dispersed nor convectively-controlled and thus prohibit calculation of Pe by asymptotic, closed-form solution of the Navier-Stokes equation]. Application of Darcy's law and Poiseuille's law to a theoretical treatment of viscous fluid flow presented by Marshall gives a simple relation between D, D_{eff}, and packed-bed porosity, ε

$$\frac{{D}_{eff}}{D} = {ɛ}^{\frac{3}{2}} . \qquad ( 10 )$$

Others report mutual diffusion coefficients for H₂ in a variety of gaseous mixtures measured by the Taylor dispersion method and fit these data to power law models with coefficients similar to those predicted by the kinetic theory of gases. From reported correlations, the following was calculated, D (973 K)=6.3±0.2 cm² s⁻¹ giving, as an absolute lower-bound, Pe≥0.3 using Eq. (7). Taking ε=0.35, a typical value for packed bed columns, Pe was found to be 1.30±0.2 under reaction conditions conforming with the balance of convective and diffusive time scales which the Bodenstein number and aspect ratio suggest.

To corroborate approximations of Pe from theoretical calculation, Pe was measured by both step-change and pulse input of H₂ through an inert bed of quartz sand particles sieved to identical size and packed to identical bed-length compared to 1.2 g MoCx/H-ZSM-5 catalyst beds used in methane DHA reactions. Pulse and step-change tracer experiments were performed changing reactor diameter (0.68-1.05 cm), temperature (303˜973 K), and total flow-rate (17.6-40 cm³ s⁻¹) to note dependence of D_{eff} solely on temperature. Following a formulism presented by Levenspiel, residence-time (exit-age) distribution, E, and mean residence time, t_{avg}, was calculated from effluent H₂ pressure histories for both pulse and step-change inputs. E is the distribution of exit ages of fluid elements which elute from the flow vessel (packed-bed reactor) and is defined as a normalized quantity such that ∫₀^{∞}Edt=1. Profiles of non-dimensional exit-age distribution

E₀+t_{avg}E, (11)

as a function of non-dimensional time elapsed from tracer entry

$$θ = \frac{t}{{t}_{avg ,}} \qquad ( 12 )$$

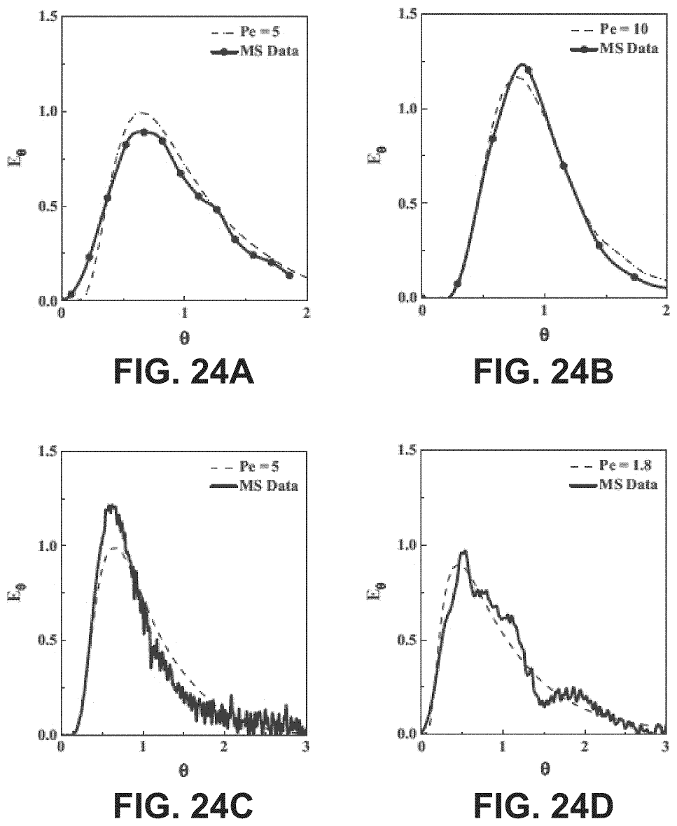

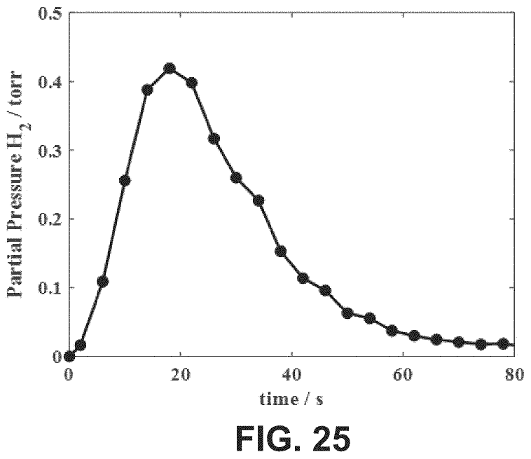

are compared to analytical solutions of differential mole balances for a perfect pulse (i.e. Dirac-delta function) or perfect step-change (i.e. Heaviside function) of inert gas passing through an axially-dispersed tubular reactor (see Supporting Information Section S3) with Pe fit by mean-square error minimization, as shown in FIGS. 24A-24D. FIG. 25 provides a representative example of tracer response of H₂ impulse input to catalyst proxies.

Table 4 collates D_{eff} and Pe measured by H₂ tracer studies and calculated using Eq. (7). Péclet numbers found from fit to experimental tracer response curves range from 1.8 to 10 depending on reactor diameter, total flow-rate, and bed temperature. D_{eff} calculated from Pe per Eq. (7), 1.4-1.5 cm² s⁻¹ at 973 K, agree well with theoretical value of 1.5 cm² s⁻¹ from Eq. (10) using ε=0.35. Taking an average experimental value D_{eff}=1.43 cm² s⁻¹ at 973 K gives Pe=1.32 at temperature and flow-rate relevant to methane DHA reactions. Péclet numbers near unity dictate a balance of convective and diffusive mass transport in tubular flow reactors and demonstrate the presence of axial dispersion phenomena in studied polyfunctional reaction configurations.

3.3. Reaction-Transport Model.

A 1-dimensional (1D) reaction-transport model is presented for a steady-state axially-dispersed packed-bed reactor inclusive to all MoCx/H-ZSM-5+Zr formulations. Methane DHA reactions and H₂ absorption are treated as pseudo-homogenous processes in catalyst and absorbent beds, respectively. Methane conversion solely to benzene and naphthalene was considered, which together constitute ≥92% carbon selectivity. Benzene+naphthalene (B+N) yield in reaction-transport simulations is thus identical to methane conversion and permits comparison to observed rates without complications conferred by methane conversion to entrained carbon and/or coke. Differential mole balance in the catalyst bed yields non-dimensional equations:

$$\begin{matrix} \frac{1}{Pe} \frac{{d}^{2} {y}_{M}}{{dx}^{2}} - \frac{{dy}_{M}}{dx} + {Da}_{B} {y}_{{CH}_{4}}^{0.72} ( 1 - {η}_{B} ) + {Da}_{N} {y}_{{CH}_{4}}^{0.74} ( 1 - {η}_{n} ) = 0 & ( 13 ) \\ \frac{1}{Pe} \frac{{d}^{2} {y}_{{H}_{3}}}{{dx}^{2}} - \frac{{dy}_{{H}_{2}}}{dx} - \frac{3}{2} {Da}_{B} {y}_{{CH}_{4}}^{0.72} ( 1 - {η}_{B} ) - \frac{8}{5} {Da}_{N} {y}_{{CH}_{4}}^{0.74} ( 1 - {η}_{N} ) + {Da}_{Zr} {Y}_{{H}_{2}} = 0 & ( 14 ) \\ \frac{1}{Pe} \frac{{d}^{2} {y}_{B}}{{dx}^{2}} - \frac{{dy}_{E}}{dx} - \frac{1}{6} {Da}_{B} {y}_{{CH}_{4}}^{0.72} ( 1 - {η}_{B} ) = 0 & ( 15 ) \\ \frac{1}{Pe} \frac{{d}^{2} {y}_{N}}{{dx}^{2}} - \frac{{dy}_{N}}{dx} - \frac{1}{10} {Da}_{N} {y}_{{CH}_{4}}^{0.74} ( 1 - {η}_{N} ) = 0 & ( 16 )\end{matrix}$$

where yⱼ is dimensionless gas-phase partial pressure, normalized by 1 bar, of component j, x is dimensionless length, and subscripts M, H₂, B, and N, refer to methane, hydrogen, benzene, and naphthalene respectively. Mole balance in the absorbent beds staged upstream and/or downstream of catalyst gives

$$\begin{matrix}\frac{1}{{Pe}_{Zr}} \frac{{d}^{2} {y}_{M}}{{dx}^{2}} - \frac{{dy}_{M}}{dx} = 0 & ( 17 ) \\ \frac{1}{{Pe}_{Zr}} \frac{{d}^{2} {y}_{M}}{{dx}^{2}} - \frac{{dy}_{M}}{dx} + {Da}_{{Z}_{r}} {y}_{{H}_{2}} = 0 & ( 18 ) \\ \frac{1}{{Pe}_{Zr}} \frac{{d}^{2} {y}_{B}}{dx} - \frac{{dy}_{B}}{dx} = 0 & ( 19 ) \\ \frac{1}{{Pe}_{Zr}} + \frac{{d}^{2} {y}_{N}}{{dx}^{2}} - \frac{{dy}_{N}}{dx} = 0 & ( 20 )\end{matrix}$$

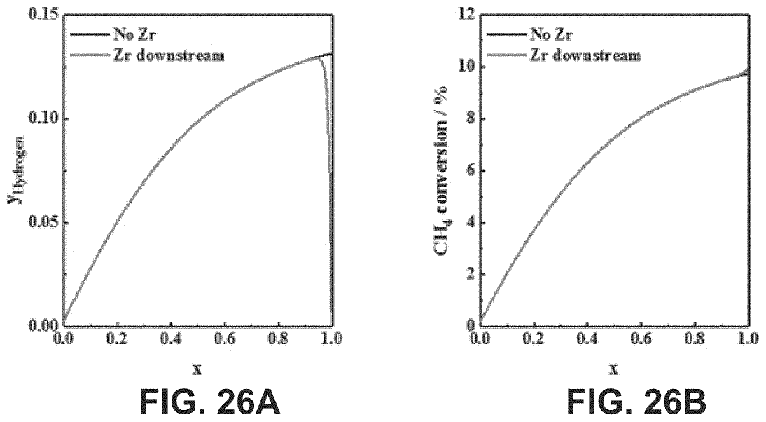

The final term in Eq. (14) is only included in interpellet mixtures wherein Eqs. (17)-(20) are unnecessary. Eqs. (13)-(20) collapse to those of an ideal PFR in the limit Pe→∞ in which bulk convection is the dominant mode of mass transport (see Supporting Information Section S4, FIGS. 26A-26B).

Non-dimensionalization of constitutive mass balance equations for a packed-bed reactor gave a rise to dimensionless parameters:

$$\begin{matrix}{Da}_{B} = \frac{{k}_{B} L}{u} & ( 21 ) \\ {Da}_{N} = \frac{{k}_{N} L}{u} & ( 22 ) \\ {Da}_{Zr} = \frac{{k}_{Zr} L}{u} & ( 23 ) \\ Pe = \frac{uL}{{D}_{eff}} & ( 24 ) \\ {Pe}_{Zr} = \frac{uL}{{D}_{eff} \vert} & ( 25 )\end{matrix}$$

where u is linear velocity, L is the length of the catalyst bed, D_{eff} is effective diffusivity, and k_{B}, k_{N}, and k_{Zr} are 1st order rate constants for the benzene synthesis reaction, naphthalene synthesis reaction, and H₂ absorption, respectively.

Rate equations for forward rates of benzene and naphthalene synthesis are determined by kinetic measurements which rigorously account for influence of H₂, C₆H₆, and C₁₀H₈ partial pressures on reversibility of methane pyrolysis reactions (Section 3.2.1).

Axial changes in total molar flow-rate, arising from stoichiometric imbalance of aromatization reactions and in-situ removal of H₂, was assessed by alteration of total pressure, P; Da and Pe remain axially invariant. Axial changes in total pressure are, at maximum, 15% and are typically ˜5-10% during methane DHA reactions as predicted by simulation and confirmed by GC analysis of reactor effluent.

Da and Pe⁻¹ reflect ratios of the characteristic convective length-scale to kinetic and diffusive length-scales, respectively. Pe and Pe_{Zr} are Péclet numbers in the catalyst and Zr bed, calculated using D_{eff} measured by H₂ tracer pulse and step-change experiments (Section 3.2.2). Da_{B} and Da_{N}, both order unity, are Damköhler numbers for forward synthesis rates of benzene and naphthalene. H₂ removal by Zr is transport-limited under reaction-relevant process flow conditions, evidenced by breakthrough curves in H₂ uptake experiments which resemble Heaviside functions. Thus, kinetic measurement of intrinsic first-order absorption rate constants, k_{Zr}, is inaccessible in current reactor configurations. Da_{Zr}>10⁴ give essentially invariant axial methane conversion and H₂ pressure profiles and are in quantitative agreement with net synthesis rates (Section 3.4). Others have reported activation energies and pre-exponential factors for first-order hydrogen absorption determined by dehydriding pressure-buildup experiments and thermogravimetric studies which give Da_{Zr}˜10⁶, conforming with Da_{Zr}>10⁴ per results of reaction-transport simulations.

Danckwerts boundary conditions are applied at both bounds of the catalyst and absorbent beds.

$$\begin{matrix}\frac{1}{{Pe}^{+}} \frac{{dy}_{j}}{dx} {\vert}_{{x}_{entrance}^{+}} = {y}_{j} ( {x}_{entrance}^{+} ) - {y}_{j} ( {x}_{entrance}^{+} ) & ( 26 ) \\ \frac{1}{Pe} \frac{{dy}_{j}}{dx} {\vert}_{{x}_{exit}} = 0 & ( 27 ) \\ {y}_{j} ( {x}_{cat / abs}^{+} ) = {y}_{j} ( {x}_{cat / abs}^{+} ) & ( 28 ) \\ \frac{1}{{Pe}^{+}} \frac{{dy}_{j}}{dx} {\vert}_{{x}_{cat / abs}^{+}} = \frac{1}{{Pe}^{+}} \frac{{dy}_{j}}{dx} {\vert}_{{x}_{cat / abs}^{+}} & ( 29 )\end{matrix}$$

where x_{entrance} refers to inlet of the catalyst or absorbent bed furthest upstream, xₑₓᵢₜ refers to outlet of the catalyst or absorbent bed furthest downstream, and x_{cat/abs} refers to bounds between catalyst and absorbent. Eqs. (26) and (27), generic “closed” Danckwerts boundary conditions, account for discontinuous change in dispersion length-scales between entrance and reaction/absorption sections and reaction/absorption and exit sections, respectively. It was presumed flow lines upstream and downstream of the reactor were dominated by convective flow such that Péclet numbers in entrance and exit sections far exceed those in reaction and absorption sections. Eqs. (28) and (29), generic “open” Danckwerts boundary conditions, demand continuity of mass and of diffusive flux between catalyst and absorbent beds which are of comparable length and thus are characterized by similar Péclet numbers.

Numerical simulation of the reaction-transport model, Eqs. (13)-(20), with appropriate boundary conditions, Eqs. (26)-(29), provides a useful tool to understand observed proximity effects in polyfunctional reaction configurations by inspection of axial product profiles otherwise unavailable by experimental means.

3.4. Measurement and Simulation of CH₄ Aromatization with In-Situ Hydrogen Abstraction.

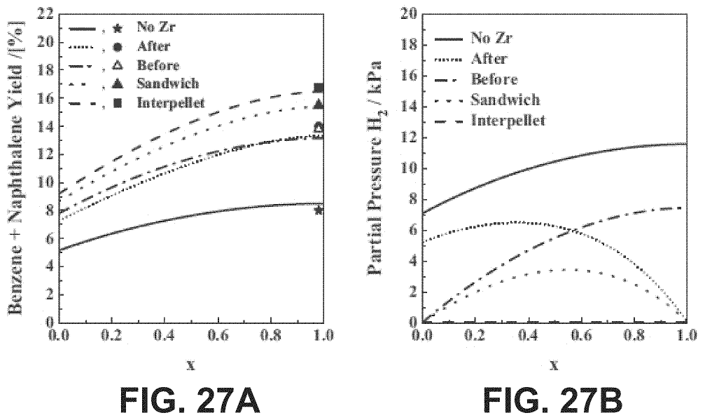

FIGS. 27A-27B compares the results of the reaction-transport model with measured benzene+naphthalene (B+N) yield histories during methane DHA reactions in all five studied reactor configurations (Section 3.1). Enhancement in instantaneous B+N yield upon zirconium addition mirrors improvements in methane conversion and cumulative aromatic yield shown in FIG. 18D; increase of B+N yield trends with proximity of Zr metal and MoCx/H-ZSM-5 catalyst. Transient loss of activity in methane pyrolysis, evident by decreasing aromatic synthesis rates, arises from both MoCx/H-ZSM-5 deactivation and zirconium saturation and is not treated in the steady-state transport model. Maximum instantaneous B+N yield coincides with a steady-state kinetic regime prior to onset of catalyst deactivation, as demonstrated by several previous investigations. Calculated B+N yield from steady-state reaction-transport simulations agree well with experimental maxima, demonstrating utility of Eqs. (13)-(20).

Maximum possible single-pass B+N yield over MoCₓ/H-ZSM-5 catalysts, henceforth referred to as the kinetic limit, is achieved in the complete absence of H₂ (i.e. η→∞) and is limited solely by reaction kinetics and contact-time. The kinetic limit, ˜17% for all configurations in FIG. 18D, is determined by simulation of Eqs. (13)-(20) with Da_{B}=0.15 and Da_{N}=0.03 and η=0 fixed for all reactions.

In all formulations, addition of Zr enhances methane conversion to benzene+naphthalene to near the kinetic limit, ˜17%. Interpellet physical mixtures provide moderate improvements to maximum single-pass B+N yield compared to layered formulations, which yield similar aromatic production in either downstream or upstream packing of Zr. Similarity of effluent aromatic yield in staged configurations is reflected in symmetry of axial H₂ profiles; equivalent removal of H₂ at low or high contact times manifests equivalent enhancement to aromatic synthesis. Partitioning zirconium in “sandwiched” configurations macroscopically mimics interpellet mixtures, improving B+N yield compared to singularly layered formulations; interpellet physical mixtures correspond to the limit of an infinite number of partitioned/stratified catalystabsorbent layers.

Non-zero B+N yield and H₂ pressure at reactor inlet in certain configurations arises from “closed” Danckwerts boundary conditions, Eqs. (26) and (27), which demand discontinuous change in species concentration from x⁺_{entrance} to x⁻_{entrance} as dispersion considerations become relevant upon transition from upstream flow lines, wherein Pe→∞, to the finite reaction and/or absorption section. Interpellet mixtures of zirconium and MoCx/H-ZSM-5 beget near-complete removal of hydrogen owing to proximity of catalyst and absorbent. Response of H₂ axial profiles throughout the catalyst bed in staged configurations results from balance of convective and diffusive length scales (Pe˜1) which permits motility of gas phase hydrogen in response to chemical potential gradients set by favorable thermodynamics of metal hydride formation. Axial H₂ pressures rise with distance from staged Zr beds as diffusional requirements rapidly increase (L_{diffusion}˜(Δx)²), preventing complete removal of hydrogen seen in interpellet mixtures. Staging zirconium before and after MoCx/H-ZSM-5, at identical mass loading, shortens average separation between catalyst and absorbent and results in suppressed H₂ pressures throughout the catalyst bed. Ability of Zr upstream of MoCₓ/H-ZSM-5 to scavenge H₂ is conferred by “open” Danckwerts boundary conditions, Eqs. (28) and (29), which enables back-mixing into the absorbent bed.

Observed aromatization rates and simulated axial B+N yield and H₂ pressure profiles demonstrate synergistic interaction between MoCx/H-ZSM-5 and Zr succeeds in influencing reversibility of methane DHA reactions despite bed-scale separation of catalyst and absorbent in layered formulations, suggesting catalyst absorbent proximity is crucial only to the extent that physical rate processes convey intermediates (e.g. H₂) between the absorbent and catalytic functions at time scales similar in magnitude to bulk advection and characteristic kinetic rates (i.e. Pe≤Da≈1).

3.5. Control of Diffusive and Convective Length-Scales.

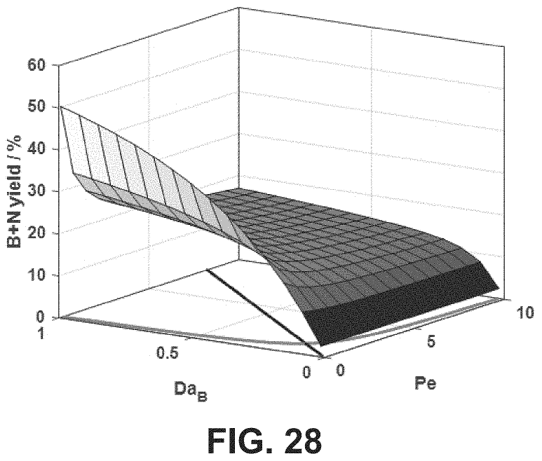

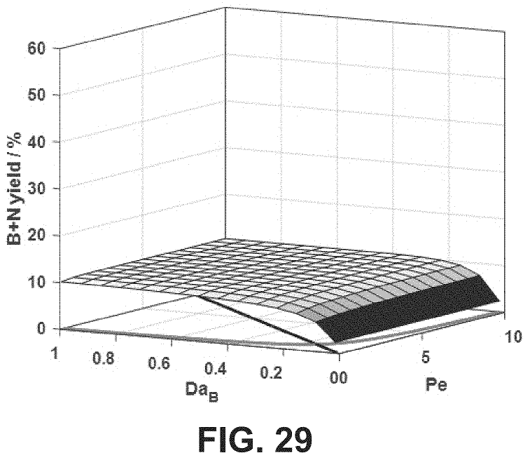

The impact of convective and diffusive length scales on methane conversion was investigated in the steady-state catalytic regime by (i) change of catalyst bed-length (equivalently catalyst mass loading) and (ii) change of linear velocity (equivalently total flow rate) in reactor configurations with Zr packed downstream of MoCₓ/H-ZSM-5 at identical catalyst loading, reaction temperature, and reactor diameter. Systematic modification of Da and Pe⁻¹, by change of L (L/Lₒ=0.2-1.6) and u (u/uₒ=1-8), provides a stringent test of the presented reaction-transport model and elucidates the key role of diffusive transport in polyfunctional staged-bed formulations. Direct influence of Da and Pe was assessed by use of the reaction-transport model; FIGS. 28-29 shows simulated surface plots of B+N yield as a function of Da and Pe with and without Zr packed downstream of catalyst.

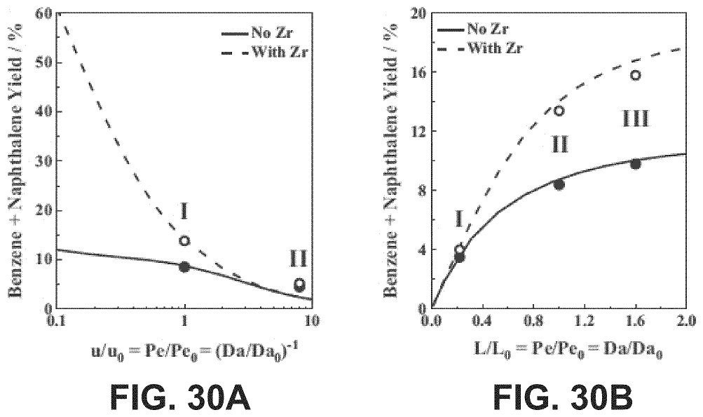

FIGS. 30A-30B compares measured maximum single-pass (i.e. steady state) benzene+naphthalene yield during methane dehydroaromatization reactions with and without Zr packed downstream of MoCx/H-ZSM-5 to those predicted by reaction simulations at varying total flow rate and catalyst loading. Data I in FIG. 30A and data II in FIG. 30B, single-pass B+N yield at u/u₀=1 and L/L₀=1, respectively, correspond to experimental measurements presented in FIGS. 27A-27B.

Decrease of u/u₀ from 8 to 1, data II to data I in FIG. 30A, enhances B+N yield from ˜4% to 10% without Zr and 14% with Zr packed downstream of the catalyst. For u/u₀≥4, B+N yield is unchanged by Zr addition, as evidenced by data II, owing to characteristic convective rates dominating over both kinetic and diffusive rates (i.e., both Da and Pe⁻¹→0). Simulation of the reaction-transport model captures the observed trends and predicts most significant influence of Zr addition to occur as u/u₀→0. Reduction of linear velocity, u, increases both Da and Pe⁻¹, simultaneously lifting kinetic and thermodynamic barriers to methane conversion in catalyst absorbent mixtures. Enhancement to kinetic limits to aromatic production, determined by Da=kL/u=kτ, results from elongated contact-times. Increase of Pe⁻¹=D/(uL) with decreasing u reflects decrease in characteristic rates of axial convection and permits improved motility of gas-phase H₂ to the downstream absorbent bed, thereby lifting thermodynamic limitations to methane DHA. Without staging of zirconium, decreasing flow rate modestly increases methane conversion until approach to the unchanged equilibrium limit (˜10%); improved kinetic limits cannot be achieved without in-situ H₂ removal.

Data I-III in FIG. 30B demonstrate that, with and without a subsequent Zr bed, increasing catalyst bed-length/mass loading monotonically enhances B+N yield. Curves generated by simulation of the reaction-transport model follow data I-III and show increase of B+N yield with L/L₀ is roughly linear at low loadings and plateaus as L/L₀→∞. Limits to B+N yield at L/L₀→∞ are ˜17% with downstream Zr and ˜10%, the equilibrium limit, without Zr addition (FIG. 29). Difference in B+N yield with and without Zr grows with L/L₀ as net synthesis rates in monofunctional formulations become more limited with increasing methane conversion. Curvilinear dependence of B+N yield with respect to catalyst loading is similar to enhancement of methane conversion, X, in an ideal PFR wherein, for a 1st-order reaction, X=(1+Da⁻¹)⁻¹. Congruence of conversion versus bed-length profiles with and without Zr is owed to converse dependence of Da and Pe⁻¹ upon bed-length, L. The disproportionate dependence of contact-time (τ=L/u) and characteristic diffusional time-scale (L²/D) upon L dictates Pe⁻¹ decreases with catalyst loading despite increase of τ and Da. Thus, systematic change of L results in a balance between kinetic and thermodynamic barriers: increasing bed-length imposes stricter limits on H₂ motility as kinetic maxima of methane conversion grow asymptotically with Da=kL/u.

3.6. Impacts of Catalyst-Absorbent Proximity on MoCx/H-ZSM-5 Deactivation.

Apparent loss of methane DHA activity in polyfunctional mixtures is complicated by conflation of dynamics attributable to catalytic CH₄ activation and stoichiometric absorption of H₂ into bulk Zr. As a metric for deactivation, decay in benzene yield, concomitant with decrease in total methane conversion, was considered, which resulted from (i) a deactivation of the MoCx/H-ZSM-5 catalyst and (ii) gradual saturation of Zr hydrogen absorption capacity. Others have demonstrated the kinetics of deactivation on conventional Mo(Cx)/H-ZSM-5 catalysts are well-described by a first-order deactivation rate constant, which assumes decrease in activity arises solely from a loss of catalytic active sites, S, at a rate

$$\frac{dS}{dt} = - {k}_{d} S . \qquad ( 30 )$$

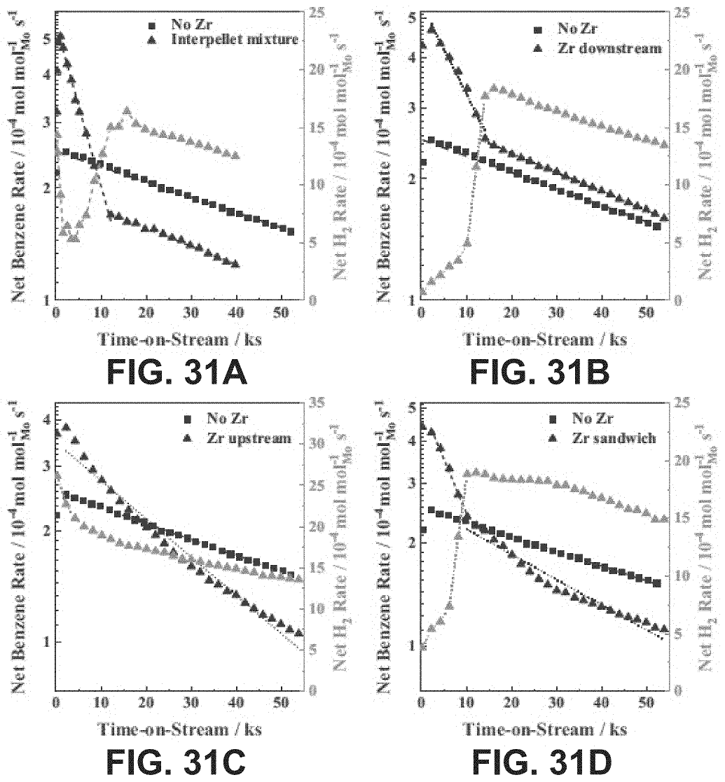

FIGS. 31A-31D shows this simple model quantitatively captures decay of net benzene formation rates for MoCx/H-ZSM-5 with or without Zr. Formulations wherein zirconium is either physically mixed with MoCx/H-ZSM-5 or staged downstream of the catalyst show two distinct deactivation regimes whose transition coincides, in each case, with hydrogen breakthrough (i.e., complete saturation of ZrHₓ). Apparent deactivation due to Zr saturation during in situ H₂ removal is more rapid in interpellet mixtures than in staged configurations owing to catalyst-absorbent intimacy increasing rates of both H₂ production by DHA reactions and absorption by proximate Zr. Following hydrogen breakthrough, net benzene rates in physical interpellet mixtures are significantly less than those intrinsic to MoCx/H-ZSM-5 catalysts suggesting increased carbon deposition during in-situ H₂ removal alters either the quantity or nature of catalytic active sites. In contrast, staging Zr downstream of MoCx/H-ZSM-5 (FIG. 31B) has little effect on benzene rates after zirconium saturation, demonstrating separation of catalyst and absorbent moderates coke formation during polyfunctional catalysis, preventing significant alteration to speciation and number of active sites. Further, first-order deactivation rate constants after hydrogen breakthrough in both interpellet and staged configurations are unchanged compared to MoCx/H-ZSM-5 catalysts under identical reaction conditions (Table 5), evincing in-situ H₂ removal in these formulations does not irreversibly alter kinetics of deactivation phenomena despite impacting carbon dynamics during enhanced methane conversion.

FIG. 31C shows benzene net synthesis rate histories for Zr packed upstream of MoCx/H-ZSM-5. Saturation of ZrHₓ upstream of MoCx/H-ZSM-5 catalyst demands H₂ transport opposite to convective flow, concomitantly eliminating both breakthrough behavior of H₂ elution curves and the two distinct deactivation regimes characteristic of polyfunctional formulations in FIGS. 31A-31B. Loss of activity in FIG. 31C follows a 1st-order deactivation profile throughout the lifetime of the catalyst and the absorbent with greater k_{d} than that intrinsic to MoCx/H-ZSM-5 (Table 5). It was posited that the high concentration of H₂ removal sites at low contact times is responsible for the rapid deactivation in these reactor configurations. MoCx/H-ZSM-5 deactivation may, in part, result due to molecular moieties formed at low contact times (equivalently low conversions) whose formation is promoted by removal of molecular hydrogen. At low conversions, H₂ abstraction may disproportionately enhance formation of graphitic carbon, CH₄→C₍ₛ₎+2H₂, which provides an absolute upper-bound on H₂ liberated per carbon, and thus, if equilibrium limited, is most aided by hydrogen removal.

FIG. 31D demonstrates H₂ breakthrough and benzene rate histories are complicated by partition of Zr upstream and downstream of the catalyst bed. Two deactivation regimes before and after H₂ breakthrough were identified; however, the transition is not distinct as in FIGS. 31A-31B. Apparent breakthrough in FIG. 31D corresponds solely to complete saturation of Zr partitioned downstream of catalyst; saturation of Zr packed upstream of catalyst is not coincident with H₂ breakthrough, as evidenced by FIG. 31C. In contrast to interpellet mixtures and formulations with Zr staged solely downstream of catalyst, first-order deactivation rate constants after H₂ breakthrough for sandwich configurations are significantly larger than those without zirconium suggesting H₂ removal sites concentrated upstream of the catalyst bed continue to deleteriously impact stability of methane aromatization rates. Disparate impact of H₂ removal at low or high contact times on catalyst stability, even after Zr saturation, supports hypotheses that graphitic carbon is involved in deactivation of active sites in Mo/H-ZSM-5 catalysts.

In summary, introduction of a continuous hydrogen scavenging function via interpellet and staged fixed-bed configurations of MoCx/H-ZSM-5 and Zr metal circumvents intrinsic thermodynamic limitations of methane DHA at 973 K, thereby, increasing single-pass methane conversion to near the kinetic limit (˜14% conversion to benzene and ˜17% conversion to benzene+naphthalene), as dictated by forward synthesis rates. Zr metal beds staggered downstream and/or upstream of the MoCx/H-ZSM-5 catalyst bed results in 1.5-1.7 fold enhancement in cumulative product yield due to transient hydrogen absorption by Zr accelerating methane conversion to aromatics. A reaction-transport model accounting for axial dispersion in packed bed reactors elucidates the underlying role of reactor hydrodynamics in lab-scale investigations. Simulated axial profiles of hydrogen and hydrocarbon partial pressure reveal Péclet number values near unity are required for efficacious removal of H₂ from the proximity of MoCx/H-ZSM-5 catalyst by staged Zr. Inert H₂ tracer studies with impulse and step-change input measure effective H₂ dispersion coefficients, D_{eff}=1.43 cm² s⁻¹, and Péclet number, Pe=1.32, to confirm the significant role of axial dispersion at process conditions relevant to catalytic studies. Both reaction-transport simulations and kinetic measurements demonstrate control of diffusive and convective length scales by alteration of catalyst bed-length and total flow-rate systematically changes methane to benzene+naphthalene yield (3%-17%), thereby explicating the critical influence of H₂ motility on efficacy of polyfunctional interaction between catalyst and absorbent. Impacts of in-situ H₂ removal on catalyst deactivation are observed to be most severe and sometimes irreversible in configurations with high concentrations of H₂ absorptive capacity at low contact times; interpellet mixtures or configurations with Zr packed solely downstream of MoCx/H-ZSM-5 have mild and reversible effects on catalyst deactivation.

Supplemental Information—S1. Hydrogen Balance for CH₄ Dehydroaromatization on MoCₓ/H-ZSM-5 and Zr Metal in Various Fixed-Bed Configurations.

The quantity of hydrogen absorbed by zirconium metal during methane DHA reactions on all on MoCₓ/H-ZSM-5 and Zr fixed-beds configurations was determined by (i) quantifying hydrogen “missing” in the effluent and (ii) performing He TPD after reaction. Corresponding results are shown in FIG. 19. The amount of hydrogen “missing” in the reactor effluent at the end of saturation of Zr metal (16.1 ks, 11.5 ks, 10.2 ks, and 10.2 ks for Zr packed downstream of MoCₓ/H-ZSM-5, Zr packed upstream of MoCₓ/H-ZSM-5, Zr packed both upstream and downstream of MoCₓ/H-ZSM-5 (sandwich), and an interpellet physical mixture of Zr and MoCₓ/H-ZSM-5, respectively) during methane reaction was estimated using Eq. (S1) and was normalized by total Zr atoms present in the catalyst bed. This number is referred as “absorbed” in FIG. 20. A temperature-programmed-desorption (TPD) in helium flow (˜0.83 cm³ s⁻¹) at ˜1193 K was performed following methane dehydroaromatization reaction at 973 K for all fixed-bed configurations and the reactor effluent was monitored using an online mass spectrometer. Hydrogen was observed to elute from the reactor presumably from the zirconium hydride formed by hydrogen absorption during methane reaction. FIG. 19 gives a representative example of He TPD on a spent Zr+MoCₓ/H-ZSM-5 interpellet physical mixture. The total amount of hydrogen eluted during TPD was calculated using helium as an internal standard. The number of hydrogen atoms eluted, normalized by the total Zr atoms present in the catalyst bed, is referred as “desorbed” in FIG. 20.

H_{missing}=(2×H₂+4×C₂Hₓ+6×C₆H₆+8×C₇H₈+10×C₈H₁₀+8×C₁₀H₈+10×C₁₀⁺)−(4×CH₄ reacted) (S1)

S2. CH₄ Dehydroaromatization on MoCₓ/H-ZSM-5 and Zr Metal in Various Fixed-Bed Configurations.

Cumulative methane conversion and product yields during methane DHA reactions were compared at 973K on five fixed-bed configurations of MoCₓ/H-ZSM-5 and Zr. FIG. 21A-21C compares methane conversion and benzene and naphthalene yield up to 20 ks TOS. FIG. 22 compares product selectivities of effluent aromatics and C₂Hₓ hydrocarbons during methane DHA on all configurations and demonstrates introduction of Zr has little effect on product distribution, suggesting prevailing dehydrogenation and cyclization events remain unperturbed by in-situ hydrogen removal.

S3. Elaboration on Measurement of Péclet Number by Tracer Response Curves.

Section 3.1.2 describes measurement of Péclet number by step-change and impulse of H₂ tracer through an inert quartz sand bed. In this supplemental section, equations necessary for calculation of residence-time distribution are detailed and provide an example of tracer response curves. Formalism summarized in Levenspiel finds residence time distribution, E, from concentration-time curves of H₂ elution. Integrating the concentration of H₂ resultant from impulse, Cₚᵤₗₛₑ, over duration, t, gives exit-age distribution

$$E = \frac{{C}_{pulse}}{{\int }_{0}^{∞} {C}_{pulse} dt} \qquad ( S2 )$$

Residence-time distribution is non-dimensionalized by mean duration of the Cₚᵤₗₛₑ curve

$${t}_{avg} = \frac{{\int }_{0}^{∞} {tC}_{pulse} dt}{{\int }_{0}^{∞} {C}_{pulse} dt} \qquad ( S3 )$$

as stated in Section 3.2.2

E_{θ}=t_{avg}E (11)

Non-dimensional duration is

θ=t/t_{avg}. (12)

Considering flow vessels with closed boundary conditions, as was done in for the reaction system, tracer concentration-time curves resultant from step-input, Cₛₜₑₚ, are transformed to E curves by Eq. (S4)

$$E = \frac{1}{{C}_{max}} \frac{{dC}_{step}}{dt} , \qquad ( S4 )$$

where Cₘₐₓ is the tracer maximum concentration following step-change input. FIG. 25 provides a representative illustration of a Cₚᵤₗₛₑ curve corresponding to E_{θ} curve in FIG. 24A in Section 3.2.2.
E_{θ} curves are fit to predictions of an unsteady differential mole balance of an axially dispersed tubular reactor

$$\frac{1}{Pe} + \frac{{d}^{2} C}{{dx}^{2}} - \frac{dC}{dx} = \frac{dC}{d θ} , \qquad ( S5 )$$

where C is non-dimensional concentration of tracer at a position x along the inert bed and time θ. Applying closed-closed boundary conditions with initial condition C(x, θ=0)=0 gives

$$\begin{matrix}{E}_{θ} = \frac{C ( 1 , θ )}{{\int }_{0}^{∞} C ( 1 , θ ) d θ} , & ( S6 ) \\ where & \\ {E}_{θ} = {e}^{\frac{Pe}{2}} {\sum }_{i = 1}^{∞} {( - 1 )}^{i + 1} \frac{8 {α}_{i}^{2}}{4 {α}_{i}^{2} + 4 Pe + {Pe}^{2}} {e}^{- \frac{θ ( {Pe}^{2} + {α}_{i}^{2} )}{4 Pe}} , & ( S 7 )\end{matrix}$$

and eigenvalues, αᵢ, are found by numerical solution to transcendental equation

$$tan ( {α}_{i} ) = \frac{4 {α}_{i} Pe}{4 {α}_{i}^{2} - {Pe}^{2}} . \qquad ( S8 )$$

Solution to Eqs. S5-S7 are used for fits to measured E curves shown in FIGS. 24A-124D.

S4. Simulation of CH₄ Aromatization with In-Situ Hydrogen Abstraction for Ideal Plug Flow Reactor.

The reaction-transport model presented in Section 3.3 was used to predict the hydrogen partial pressure profile and methane single-pass conversion along the axial coordinate of the catalyst bed for an ideal plug flow reactor with Pe=100. The results of the simulation are shown in FIGS. 26A-26B. The predominance of convective mass transport as Pe→∞ or Pe>>Da limits the capacity of neighboring fluid elements to transfer matter axially, preventing bed-scale suppression of H₂ partial pressure (FIG. 26A) and enhancement in methane conversion (FIG. 26B).

S5. Calculation of H₂ Mutual Diffusion Coefficients from Correlative Relations.

Others reported mutual diffusion coefficients of dilute H₂ in air, N₂, and O₂ at atmospheric pressure over a wide range of temperature (303 K-503 K). Data are fit to correlations resembling power-law dependences predicted by the kinetic theory of gases

D=AT^{B} (S9)

where A and B are fitting parameters listed below.

S6. Reaction-Transport Simulations: Effects of Damköhler and Péclet Number.

Section 3.5 examines the key interplay of kinetic, convective, and diffusive length-scales in catalyst-absorbent mixtures for methane DHA by systematic change of catalyst bed-length, L, and linear velocity, u. FIGS. 28-29 are surface plots of B+N yield as a function of Da_{B} and Pe, with and without Zr, respectively. Da_{B} and Da_{N} are increased in concert such that the condition Da_{B}/Da_{N}=k_{B}/k_{N} holds for all values of Da_{B}. FIG. 29 demonstrates that, without introduction of a hydrogen removal function, increase of Da or Pe⁻¹ is unable to effect enhancement of B+N yield or methane conversion beyond limits prescribed by equilibrium. Addition of Zr in FIG. 28 permits rapid increase of B+N yield as kinetic limits and resistance to dispersive H₂ transport are relaxed with increase of Da and Pe⁻¹, respectively.

TABLE 4
Peclet number, Pe, and effective diffusion coefficients, D_{eff},
determined from fit to H₂ tracer responses using Eqs. 11 and 12 and
calculated from correlation 28 and Eqs. 7 and 10.

| | | | Reactor | | |
|---|---|---|---|---|---|
| | Temperature | Flow Rate | Diameter | | D_{eff}^{b} |
| Method | [K] | [cm³ s⁻¹] | [cm] | Pe^{b} | [cm² s⁻¹] |
| H₂ Pulse in | 973 | 20.0 | 0.68 | 5.0 | 1.4 |
| CH₄ (FIG. | | | | | |
| 24A) | | | | | |
| H₂ Pulse in | 973 | 40.0 | 0.68 | 10 | 1.4 |
| CH₄ (FIG. | | | | | |
| 24A) | | | | | |
| H₂ Step- | 303 | 17.6 | 1.05 | 5.0 | 0.5 (2.3^{e}) |
| Change in | | | | | |
| Argon (FIG. | | | | | |
| 24A) | | | | | |
| H₂ Pulse in | 973 | 17.6 | 1.05 | 1.8 | 1.5 |
| CH₄ (FIG. | | | | | |
| 24A) | | | | | |
| Eqs. (7) and | 973^{d} | 13.0^{d} | 1.05^{d} | 1.3^{d} | 1.5^{d} |
| (10)^{c} | | | | | |

^{a}All experiments performed at 973 K
^{b}Calculated taking ε = 0.35
^{c}D taken from correlations given by others.
^{d}Reaction conditions relevant to methane DHA reactions performed in this Example
^{e}Adjusted to 973 K assuming D_{eff} ~T^{3/2}

TABLE 5
Summary of deactivation constants for benzene calculated using Eq. 30
for MoCx-H-ZSM-5 and Zr in different fixed bed configurations.

| | k_{d} before H | |
|---|---|---|
| Fixed-bed configuration | breakthrough/s⁻¹ | K_{d} after H breakthrough/s⁻¹ |
| MoCₓ/H-ZSM-5 only | NA | 0.0094 |
| Interpellet mixture^{a} | 0.1058 | 0.0113 |
| Zr downstream^{b} | 0.0418 | 0.0109 |
| Zr upstream^{c} | NA | 0.0243 |
| Sandwich^{d} | 0.0733 | 0.0171 |

^{a}Corresponding data shown in FIG. 31A
^{b}Corresponding data shown in FIG. 31B
^{c}Corresponding data shown in FIG. 31C
^{d}Corresponding data shown in FIG. 31D

TABLE 6
Diffusion-coefficient correlation parameters from literature for
intermolecular bulk counter diffusion of H₂ and various
gases or gaseous mixtures.

| System | A/[cm²K^{-B}s⁻¹] | B |
|---|---|---|
| H₂-air | 4.19 · 10⁻⁵ | 1.73 |
| H₂—N₂ | 4.68 · 10⁻⁵ | 1.71 |
| H₂—O₂ | 3.68 · 10⁻⁵ | 1.76 |

Using the reported values gives an average diffusion coefficient D (973 K) = 6.3 ± 0.2 cm² s⁻¹ across the three studied systems.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

What is claimed is:

1. A method of producing aromatics, comprising:
contacting a methane-containing feed stream with a pre-carburized catalyst to form aromatics and hydrogen, wherein the pre-carburized catalyst is disposed within a reactor and comprises a carbidic form of an active metal supported on a proton form of a zeolite, wherein the reactor further comprises a hydrogen-accepting component that removes at least a portion of said hydrogen.

2. The method of claim 1, wherein the contacting proceeds at or to a temperature of at least about 950 K.

3. The method of claim 1, wherein the contacting proceeds under about atmospheric pressure.

4. The method of claim 1, wherein single-pass methane conversion is greater than equilibrium methane conversion under the same reaction conditions.

5. The method of claim 1, wherein an average particle size of the pre-carburized catalyst is in the range of about 180 μm to about 425 μm.

6. The method of claim 1, wherein an average particle size of the hydrogen-accepting component is in the range of about 180 μm to about 425 μm.

7. The method of claim 1, wherein the reactor is a staged and stratified reactor comprising alternating layers of the pre-carburized catalyst and hydrogen-accepting component.

8. The method of claim 1, wherein the hydrogen-accepting component is positioned upstream from the pre-carburized catalyst.

9. The method of claim 1, wherein the hydrogen-accepting component is positioned downstream from the pre-carburized catalyst.

10. The method of claim 1, wherein the hydrogen-accepting component is positioned both downstream and upstream from the pre-carburized catalyst.

11. The method of claim 1, wherein the pre-carburized catalyst and hydrogen-accepting component are disposed within the reactor as an interparticle mixture.

12. The method of claim 1, wherein the pre-carburized catalyst comprises an active metal of the formula MCₓ, where M is the active metal, C is carbon, and x is at least 0.01.

13. The method of claim 1, wherein the active metal is selected from the group consisting of molybdenum, vanadium, chromium manganese, zinc, iron, cobalt, nickel, copper, gallium, germanium, niobium, molybdenum, ruthenium, rhodium, silver, tantalum, tungsten, rhenium, platinum, or lead.

14. The method of claim 1, wherein the hydrogen-accepting component is selected from the group consisting of zirconium, titanium, niobium, tantalum, hafnium, vanadium, or zinc.

15. The method of claim 1, wherein the aromatics are formed without an induction period.

16. The method of claim 1, wherein the aromatics include one or more of benenze, naphthalene, toluene, and xylene.

17. The method of claim 1, further comprising regenerating the hydrogen-accepting component by thermal treatment under inert flow.

18. The method of claim 1, further comprising exposing, prior to the contacting, a precatalyst comprising an active metal supported on a proton form of a zeolite to methane to obtain the pre-carburized catalyst.

19. A staged and stratified catalyst for non-oxidative methane dehydroaromatization to form aromatics and hydrogen from methane, the catalyst comprising: alternating layers of a pre-carburized catalyst and a hydrogen-accepting component, wherein the pre-carburized catalyst comprises a carbidic form of an active metal supported on a proton form of a zeolite, wherein the hydrogen-accepting component absorbs at least a portion of said hydrogen.

20. An interparticle catalyst mixture for non-oxidative methane dehydroaromatization to form aromatics and hydrogen from methane, the catalyst comprising: a mixture comprising particles of a pre-carburized catalyst and particles of a hydrogen-accepting component, wherein the pre-carburized catalyst comprises a carbidic form of an active metal supported on a proton form of a zeolite, wherein the hydrogen-accepting component absorbs at least a portion of said hydrogen.
